(19) United States
(12) Patent Application Publication
(10) Pub. No.: US 2024/0124410 A1
(43) Pub. Date: Apr. 18, 2024

(54) SMALL MOLECULES AND THEIR USE AS MALT1 INHIBITORS

(71) Applicants: University of Pittsburgh - Of the Commonwealth System of Higher Education, Pittsburgh, PA (US); THE REGENTS OF THE UNIVERSITY OF MICHIGAN, Ann Arbor, MI (US)

(72) Inventors: Peter C. Lucas, Pittsburgh, PA (US); Linda M. McAllister, Pittsburgh, PA (US); Heejae Kang, Pittsburgh, PA (US); Beibei Chen, Pittsburgh, PA (US); Lisa Maurer, Pittsburgh, PA (US); Dong Hu, Pittsburgh, PA (US); Jing Cheng, Pittsburgh, PA (US); Linda Klei, Pittsburgh, PA (US); Zaneta Nikolovska-Coleska, Ann Arbor, MI (US)

(73) Assignees: University of Pittsburgh - Of the Commonwealth System of Higher Education, Pittsburgh, PA (US); Regents of the University of Michigan, Ann Arbor, MI (US)

(21) Appl. No.: 18/266,525

(22) PCT Filed: Mar. 24, 2022
(86) PCT No.: PCT/US2022/021720

§ 371 Date: Jun. 9, 2023

Related U.S. Application Data
(60) Provisional application No. 63/166,051, filed on Mar. 25, 2021.

(51) Int. Cl. C07D 317/58; A61P 35/00; C07C 317/26; C07D 209/14; C07D 209/38; C07D 215/14; C07D 235/14; C07D 241/42; C07D 249/18; C07D 263/56; C07D 277/64; C07D 333/58; C07D 471/04

(52) U.S. Cl. CPC C07D 317/58; A61P 35/00; C07C 317/26; C07D 209/14; C07D 209/38; C07D 215/14; C07D 235/14; C07D 241/42; C07D 249/18; C07D 263/56; C07D 277/64; C07D 333/58; C07D 471/04

(57) ABSTRACT

Small molecule inhibitors that block the interaction between B-cell lymphoma 10 protein (BCL10) and mucosa- associated lymphoid tissue lymphoma translocation protein 1 (MALT1), thereby inhibiting both the protease and scaffolding activities of MALT1, and MALT1- dependent downstream signaling, including IL-6 and IL-10 secretion by B-cell lymphoma cells and IL-2 transcription and secretion by Jurkat T cells.

### CROSS REFERENCE TO RELATED APPLICATIONS

This claims the benefit of U.S. Provisional Application No. 63/166,051, filed Mar. 25, 2021, which is incorporated by reference herein.

### ACKNOWLEDGMENT OF GOVERNMENT SUPPORT

This invention was made with government support under R01 HL082914 awarded by the National Institutes of Health. The government has certain rights in the invention.

### BACKGROUND

MALT1 (mucosa associated lymphoid tissue lymphoma translocation protein-1) is an intracellular signaling protein, known to activate both innate (natural killer cells NK, dendritic cells DC, and mast cells) and adaptive immune cells (T cells and B cells). The function of MALT1 is best known in the context of T cell receptor (TCR signaling), where it mediates nuclear factor k (NF-κB) signaling, leading to T cell activation and proliferation.

It is now well accepted that MALT1 acts downstream of various receptors in a complex composed of CARMA proteins (Caspase recruitment domain-containing protein also known as CARD-containing MAGUK protein), the adaptor protein BCL10 (B-Cell Lymphoma 10) and MALT1 itself. These so-called CBM (CARMABCL10-MALT1) complexes are crucial for the regulation of the classical NF-κB pathway and other biological processes. MALT1 has both a scaffold function (contributing to the assembly of other signaling complexes) and a protease function (cleaving a limited repertoire of substrate proteins). Together, these two functions of MALT1 work in concert to have an important role in the activation of the transcription factor NF-κB, in the production of interleukin-2 (IL-2) and other cytokines, and in overall T and B lymphocyte activation and proliferation.

Because of MALT1's pivotal role in NF-κ B signaling and concomitant involvement in T and B-cell activation and proliferation, pharmacological inhibitors are an attractive strategy for treating neoplastic and inflammatory diseases associated with deregulated MALT1 signaling. To date, however, compounds that have been developed and tested as MALT1 inhibitors only block one of the two actions of MALT1: the protease activity of MALT1, but not the scaffolding activity. Blocking only the protease activity of MALT1—but not both the protease and scaffold activities—has been shown to cause severe autoinflammation in mice. This is thought to be caused by residual immune activation by the preserved MALT1 scaffolding function, combined with defective peripheral tolerance and impaired regulatory T cell (Treg) function. The Tregs are a subpopulation of T cells that modulate the immune system, maintain tolerance to self-antigens by immunosuppression, and prevent autoimmune disease. Imbalanced T effector and Treg function, followed by paradoxical autoimmune-like pathology observed in MALT1 protease inhibition explains why there has been only one clinical trial for MALT1 inhibition.

### SUMMARY

Disclosed herein is a method comprising administering to a subject in need of, or has been recognized as being in need of, treatment with a MALT1 inhibitor, a therapeutically effective amount of a compound, or a pharmaceutically acceptable salt thereof, having a structure of:

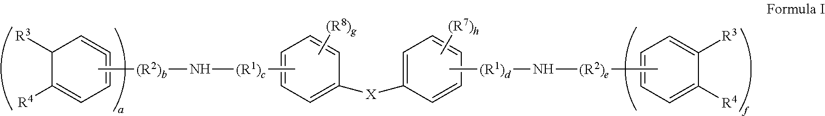

wherein X is (—CH₂—)_{y}, (—CH(R¹³)—)_{y}, (—C(R¹⁴)(R¹⁵)—)_{y}, —O—, —S(O₂)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R)¹⁰)ₓ, (—C(R¹¹)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d or e is 1; and
provided that if —(R²)_{b}— is H, then a is 0, and if —(R²)ₑ— is H, then f is 0.

Also disclosed herein is a pharmaceutical composition comprising a pharmaceutically acceptable additive and a compound, or a pharmaceutically acceptable salt thereof, having a structure of:

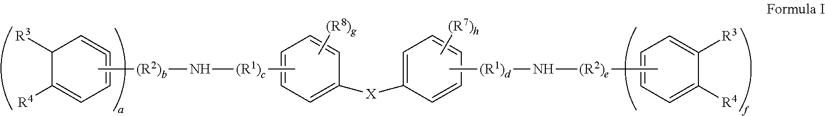

wherein X is (—CH₂—)_{y}, (—CH(R¹³)—)_{y}, (—C(R¹⁴)(R¹⁵)—)_{y}, —O—, —S(O₂)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R¹⁰)ₓ, (—C(R¹¹)(R¹²)—)ₓ, —S(O)₂—, S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R4 together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d or e is 1; and
provided that if —(R²)_{b}— is H, then a is 0, and if —(R²)ₑ— is H, then f is 0,
herein the pharmaceutical composition is in a unit dosage form.

Further disclosed herein is a compound, or a pharmaceutically acceptable salt thereof, having a structure of:

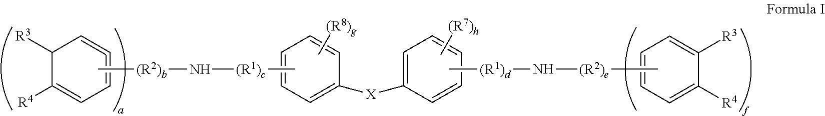

wherein X is (—CH₂—)_{y}, (—CH(R¹³)—)_{y}, (—C(R¹⁴)(R¹⁵)—)_{y}, —O—, —S(O₂)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R)¹⁴)ₓ, (—C(R¹¹)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d or e is 1; and
provided that if —(R²)_{b}— is H, then a is 0, and if —(R²)ₑ— is H, then f is 0, and provided that the compound is not

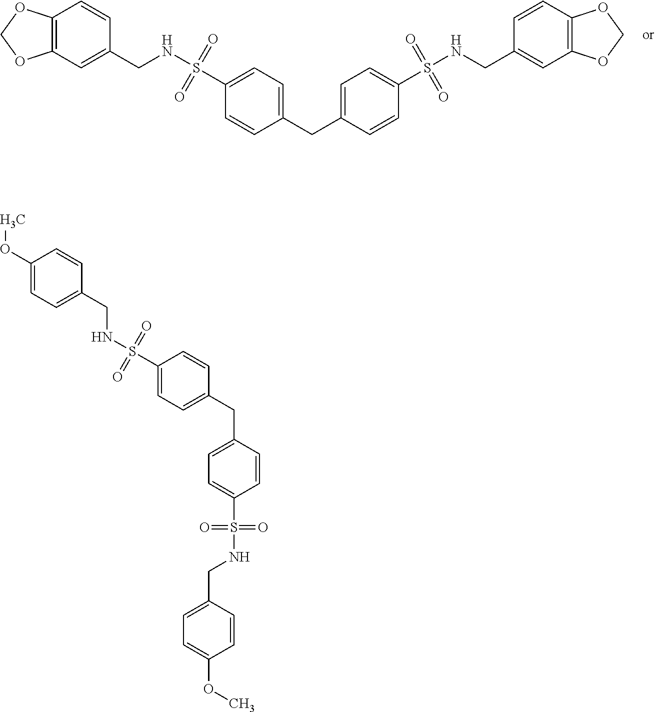 [SMILES: O=S(=O)(NCc1ccc2c(c1)OCO2)c1ccc(Cc2ccc(S(=O)(=O)NCc3ccc4c(c3)OCO4)cc2)cc1 ; COc1ccc(CNS(=O)(=O)c2ccc(Cc3ccc(S(=O)(=O)NCc4ccc(OC)cc4)cc3)cc2)cc1]

The foregoing will become more apparent from the following detailed description, which proceeds with reference to the accompanying figures.

### BRIEF DESCRIPTION OF THE DRAWINGS

FIG. 1. The MALT1 Ig1-2 domains contribute to BCL10-MALT1 interaction. (A) In order to evaluate the interaction of specific regions of MALT1 with BCL10, we performed co-immunoprecipitation analysis using HA-tagged MALT1 fragments and MYC-tagged BCL10. Both MALT1 (DD+Ig1-2) and MALT1 (Ig1-2) co-IP with MYC-BCL10 (lanes 4 and 6) whereas the MALT1 (DD) fragment demonstrates almost no interaction with MY-BCL10 on co-IP (lane 5). To control for specificity, we demonstrate that while MALT1 (Ig1-2) binds to BCL10, it does not bind to other CARD domain containing proteins, RICK or MAVS via co-IP (lanes 7 and 8). Blots are representative of at least 3 experiments. (B) SPR analysis was performed to identify specific regions within MALT1 that are required for interaction with BCL10. Results demonstrate that MALT1 (DD+Ig1-2) and MALT1 (Ig1-2) bind to BCL10, whereas MALT1 (DD) does not. Sensograms show concentration-dependent binding of MALT1 fragments, tested at increasing concentration, with full-length BCL10 immobilized on a sensor chip. K., Koff and Ka were calculated by simultaneous nonlinear regression using 1:1 binding model. Experimental data are shown in black or blue, whereas global fit analyses are shown in red. (C) Co-immunoprecipitation analysis was performed to test whether MALT1 (Ig1-2) can interact with monomeric (non-oligomerizable) BCL10. Results demonstrate that MALT1 (Ig1-2) interacts equally well with wild-type MYC-BCL10 and the non-oligomerizing, monomeric MYC-BCL10 E53R mutant. Plasmids were transfected into HEPG2 BCL10^{-/-} cells lacking endogenous BCL10 in order to prevent any contribution of endogenous BCL10 to this interaction. Western blots represent at least 3 experiments. (D) Point mutations or deletion insertion mutations with alanines were introduced into HA-MALT1 (Ig1-2) in order to evaluate the role of specific amino acid residues in the predicted binding groove within the MALT1(Ig1-2) fragment. MALT1 (Ig1-2) with specific mutations in the Ig1-2 groove demonstrate decreased ability to interact with BC110 in a Co-IP assay (lanes 4-6). Western blots represent at least 2 experiments. (E) The MALT1 V189R mutation disrupts the interaction of BCL10 both in the context of the MALT1 (Ig1-2) fragment and in the context of full-length MALT1. Western blots represent at least 3 experiments. (F) PyMol model of the groove identified within MALT (Ig1-2) that interacts with BCL10 (PDB file 3K0W). Residues implicated in MALT1's interaction with BCL10 in Co-IP analysis are shown in purple. Green represents the strands of Ig1-2 and gray is the space filling model. DD=Death Domain; Ig1-2=immunoglobulin-like 1-2 domains; IP=immunoprecipitation; IB=immunoblot; SPR=surface plasmon resonance.

FIG. 2. In silico screening identifies small molecules targeting the predicted BCL10-binding groove within MALT1 (Ig1-2). (A) We performed a structure-guided in silico screen of 3 million compounds, using the LibDock program from Discovery Studio 3.5, in order to identify candidate molecules that could potentially fit within the identified groove in the MALT1 Ig1-2 domain. Lipinski's rule of five filters (LipinskiRo5) were applied to enrich for compounds with drug-like properties. Potential compound hits were then screened for their ability to disrupt the MALT1-BCL10 interaction using SPR analysis. 9 candidate compounds were then subjected to a second, ELISA-based MALT1-BCL10 interaction assay, identifying 2 compounds that could disrupt the interaction in both SPR and ELISA assays. M1i-124 and M1i-1255 are the two compounds identified to disrupt the BCL10-MALT1 interaction. M1i-124 became our lead compound and M1i-1255 is partially characterized in FIG. 8. (B) Structure of M1i-124. (C) Model of M1i-124 docked onto the MALT1 Ig1-2 domain groove (PDB 3K0W). (D) SPR analysis demonstrates that M1i-124 (100 μM) disrupts the BCL10-MALT1 interaction. (E) SPR analysis was performed to determine the IC50 for M1i-124 disruption of the BCL10-MALT1 interaction in a dose-response assay. (F) ELISA analysis confirmed that M1i-124 disrupts the interaction between BCL10 and MALT1 in dose dependent fashion (left). Dose-dependent inhibition of BCL10-MALT1 interaction in the ELISA assay was determined using the following equation: Y=bottom+(top-bottom)/(1+10\hat{ }(X-LogIC50), with the top and bottom representing the plateaus in the units of Y axis, and with the assumption of one-site binding that is reversible and at equilibrium. M1i-124 displays dose-dependent inhibition of BCL10-MALT1 binding with an IC50 of 89 uM. In contrast, control compound V001-9748, which failed to show inhibition of the interaction by SPR, also does not display dose-dependent inhibition in the ELISA (right). (G) Structure of derivative compound, M1I-124d1. Circles highlight sites that differ from parent compound, M1i-124. (H) ELISA analysis demonstrates that M1i-124d1 disrupts the interaction between BCL10 and MALT1 with a lower IC50 as compared to parent compound. (I) ELISA analysis demonstrates that both M1i-124 and M1i-124d1 retain the ability to disrupt the BCL10-MALT1 interaction in the presence of 0.005% tween. SPR =surface plasmon resonance. ELISA =Enzyme Linked Immunoassay.

FIG. 3. M1i-124 and its derivative disrupt MALT1 protease and scaffolding function in T-cells. (A) Cartoon demonstrating lymphocyte antigen receptor stimulation leading to assembly and activation of the CARMA1-BCL10-MALT1 complex. Activated MALT1 possesses two functions: 1) MALT1 serves as a protease to cleave multiple protein substrates including RelB and N4BP1, and 2) MALT1 serves as a scaffold to recruit downstream signaling proteins that mediate phosphorylation and activation of the inhibitor of Kappa B kinase (IKK) complex, which then leads to activation of the NF-κ transcription factor. (B and C) Both M1i-124 and M1i-124d1 inhibit antigen receptor-dependent induction of MALT1 proteolytic cleavage of RelB (B) and N4BP1 (C) in Jurkat T cells. Cells were pretreated with compounds for 20 hours and then 5 μM MG132 was added for 1 hour before stimulation with phorbol 12-myristatel3 acetate and ionomycin (PMA/iono). Known MALT1 protease inhibitor, mepazine, serves as a positive and compound V001-9748 serves as a negative control. Quantification of the ratio of cleaved band/full-length band is shown in the bar graph above Western blots (mean±SEM; N=4). Statistical analysis was performed using one-way ANOVA and Dunnett test to correct for multiple comparisons. (D and E) Treatment of Jurkat T cells with compound M1i-124 (D) or M1i-124d1 (E) leads to inhibition of anti-CD3/CD28-induced phosphorylation of IKKα/β. Cells were pretreated with indicated compounds for 20 hours, then stimulated with anti-CD3/CD28 for 0, 5, 10, 15, and 20 minutes. (F and G) Neither M1i-124 (F) or M1i-124d1 (G) inhibits T cell receptor (TCR)-induced phosphorylation of ERK. Cells were treated as described above in D and E. Quantification of the ratio of phospho-IKK/total IKK (D and E) or the ratio of phospho-ERK/total ERK (F and G) is shown as bar graphs above the Western blots. Statistical analysis was performed using unpaired t-test comparing each stimulation time point (mean±SEM; N=5). (H) Compounds M1i-124 and M1i-124d1 inhibit PMA/iono-induced transcription of interleukin 2 (IL-2) in Jurkat T cells at 1 μM. (I) Compound M1i-124 (left) and M1i-124d1 (right) exhibit dose-dependent inhibition of IL-2 secretion. Statistical analysis was performed using one-way ANOVA and Dunnett test to correct for multiple comparisons. *p<0.05, **p<0.01, ***p<0.001, ****p<0.0001

FIG. 4. M1i-124 and its derivative inhibit MALT1-dependent signaling, proliferation and survival in Activated B-cell like Diffuse Large B-cell Lymphoma (ABC-DLBCL) cells. (A) M1i-124 inhibits constitutive MALT1 protease activity in ABC-DLBCL. OCI-Ly3 cells were treated overnight −/+1μM M1i-124. MG132 was added 5 hours prior to harvesting and the level of RelB cleavage was compared by Western blot. (B) M1i-124 inhibits constitutive MALT1 protease-dependent expression of interleukins IL-6 and IL-10 in ABC-DLBCL cells. OCI-Ly3 cells were treated −/+M1i-124 and IL-6 and IL-10 mRNA levels were compared using quantitative RT-PCR. Error bars represent SEM. (C and D) Both M1i-124 and M1i-124d1 inhibit IL-6 and IL-10 secretion in a concentration-dependent manner in OCI-Ly3 (C) and TMD8 (D) ABC-DLBCL cells. Cytokine secretion was measured by ELISA. (E) M1i-124 and M1i-124d1 inhibit the proliferation of ABC-DLBCL cells (OCI-Ly3 and TMD8), but not Germinal Center B-cell Diffuse Large B-cell Lymphoma (GCB-DLBCL) cells (OCI-Ly1 and OCI-Ly7). Cells were treated with a single dose of 1 μM M1i-124 or M1i-124d1 or mepazine for 12 days. Error bars represent SEM of 3-6 independent experiments. (F) Cell Trace Violet tracking dye was used to demonstrate that M1i-124 inhibits cell division in ABC-DLBCL cells (OCI-Ly3 and TMD8). M1i-124 demonstrates a lesser effect in OCI-Lyl GCB-DLBCL cells. *p<0.05, **p<0.01, ***p<0.001, ****p<0.0001. SEM=standard error of the mean. MFI=Mean Fluorescence Intensity.

FIG. 5. M1i-124 exhibits promising pharmacokinetic properties. (A) Bioavailability radar analysis was performed to predict the ‘drug-likeness’ of M1i-124. The pink area illustrates the optimal range of oral bioavailability for each physicochemical property (lipophilicity, size/molecular weight, polarity, solubility, saturation and flexibility). To be estimated as drug-like, the red line should ideally be included within the pink area. (B) In vivo pharmacokinetic analysis in mice demonstrates that following either oral or IV administration, M1i-124 demonstrates a plasma terminal half-life of approximately 8.7 hours. (C) Metabolic clearance of M1i-124 was evaluated by in vitro microsomal stability analysis. t_{1/2} in human and mouse liver microsomes is estimated to be 5.14 minutes and 2.35 minutes, respectively. (D) Detected M1i-124 metabolites are shown, produced following microsomal incubation. (E) M1i-124 demonstrates minimal inhibition of cytochrome P450 enzymes.

FIG. 6. M1i-124 suppresses the growth of ABC-DLBCL tumor xenografts in vivo. (A and B) Treatment of mice with M1i-124 leads to significantly reduced tumor volume (A) and tumor weight (B) in a xenograft model of ABC DLBCL. NOD/SCID mice were inoculated, by subcutaneous injection into the right flank, with 10×10⁶ TMD8 cells suspended in 50% matrigel. Once tumors reached an average volume of 100 mm 3, mice were randomized to receive daily intraperitoneal (IP) injections of 50 mg/kg M1i-124 or equivalent volume of DMSO control vehicle for 12 days. One day after the last IP injection, tumors were harvested for analysis. Resected tumors from the DMSO control group (n=8) and M1i-124 treatment group (n=8) are shown in B. Weights of individual tumors are shown at right. (C) Mice treated with M1i-124 demonstrated a slightly lower average body weight compared to DMSO control-treated mice. (D) Treatment with M1i-124 also led to reduced tumor volume in a second ABC-DLBCL cell line xenograft model, OCI-Ly3. Mice were treated and tumors were analyzed as described above for A and B. Two-way ANOVA and Sidak's multiple comparison tests were performed to analyze tumor growth differences between groups. Unpaired t-test was performed to compare tumor weight and body weight. *p<0.05, *** p<0.001, **** p<0.0001.

FIG. 7. (A) Schematic of MALT1 domain structure. DD=death domain; R149 is the site of auto-proteolysis; Ig=immunoglobulin-like domain; ‘caspase-like’=proteolytic domain; C464=catalytic cysteine residue; K644=monoubiquitination site. (B) Co-immunoprecipitation analysis confirms that the BCL10 E53R mutant is unable to self-oligomerize. (C) Co-immunoprecipitation analysis was performed to identify the amino acid residues within MALT1 that are required for interaction with BCL10. MALT1 point mutations D220K and E224K may disrupt interaction with BCL10, but to a lesser extent than amino acid residues highlighted in the analogous assay shown in FIG. 1D. The MALT1 V81R mutation, which is known to disrupt the BCL10-MALT1 interaction, serves as a positive control.

FIG. 8. (A) Coomassie-stained SDS-PAGE gel of purified recombinant MALT1 fragments. DD=death domain; Ig1/2=Immunoglobulin like domains 1 and 2. (B) A schematic of the surface plasmon resonance (SPR) platform is shown. BCL10 proteins are immobilized on the sensor chip and a range of concentrations of three MALT1 domains in liquid phase are flowed over the sensor chip to detect real-time binding. (C) Schematic of ELISA-based protein-protein interaction (PPI) assay (left) and validation of the assay (right) are shown. This ELISA assay confirms the interaction of BCL10 with MALT1 whereas GRK2 serves as a negative control protein that does not interact with BCL10. (D) The structure of M1i-1255 is shown. This compound was identified as one the potential hits from the in silico screen described in FIG. 2 that could be validated to disrupt the BCL10-MALT1 interaction by both SPR and ELISA assays. (E) Bioavailability radar analysis of M1i-1255 was performed to examine the drug-like properties of this compound. The pink area illustrates the optimal range of oral bioavailability for each physicochemical property (lipophilicity, size/molecular weight, polarity, solubility, saturation and flexibility). To be estimated as drug-like, the red line should ideally be included within the pink area. (F) SPR analysis demonstrates complete inhibition of BCL10-MALT1 binding by 100 μM M1i-1255. (G) M1i-1255 shows dose-dependent inhibition of the Bc110-MALT1 interaction with an EC50 of 21.9 μM as determined by SPR analysis. (H) Dose-dependent inhibition of Bc110-MALT1 interaction by compounds in the ELISA was determined using the following equation: Y=bottom+(top-bottom)/(1+10\hat{ }(X-LogIC50), with the top and bottom representing the plateaus in the units of Y axis, and with the assumption of one-site binding that is reversible and at equilibrium. M1i-1255 displays dose-dependent inhibition of Bc110-MALT1 binding with an IC50 of 17 μM as determined by ELISA.

FIG. 9. Table summarizing the Eurofins analysis of potential off-target effects of M1i-124. EC50/1050 values above 10 μM are generally considered to be acceptable. FIG. 10. Representative hematoxylin and eosin (H&E) staining of organ sections (lung, heart, kidney, liver and adrenal) from control and M1i-124 treated mice described in FIG. 6. No histologic evidence of organ toxicity was detected on H&E in the M1i-124 treated animals.

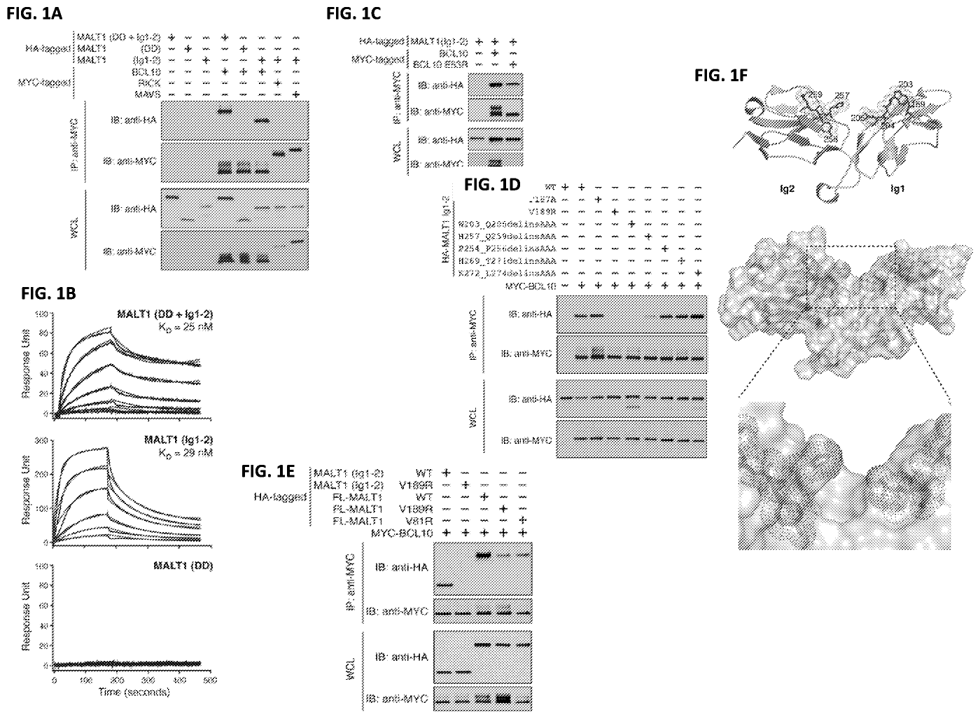

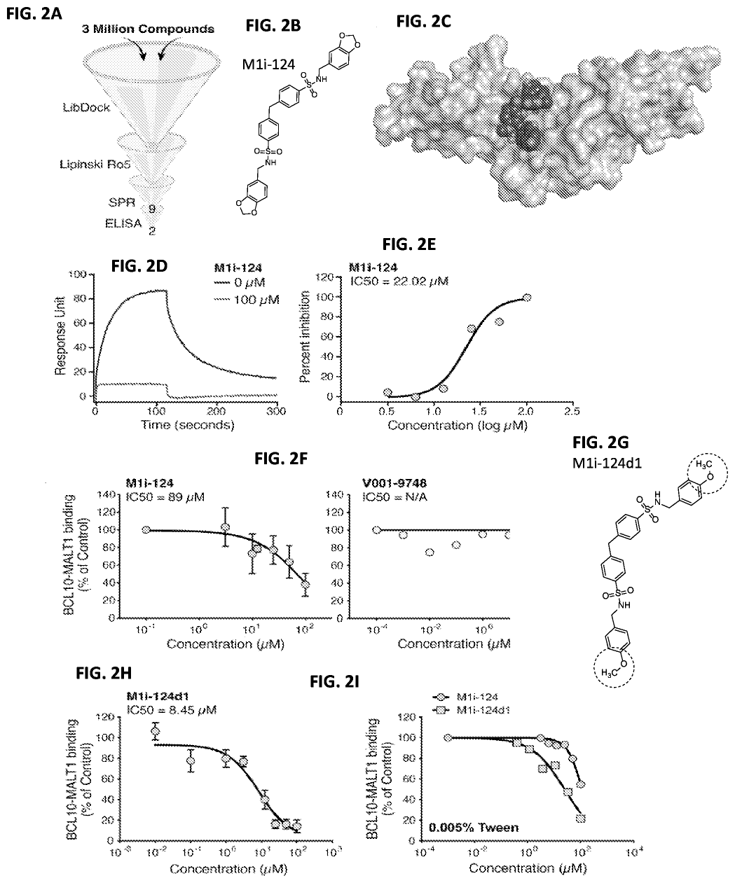

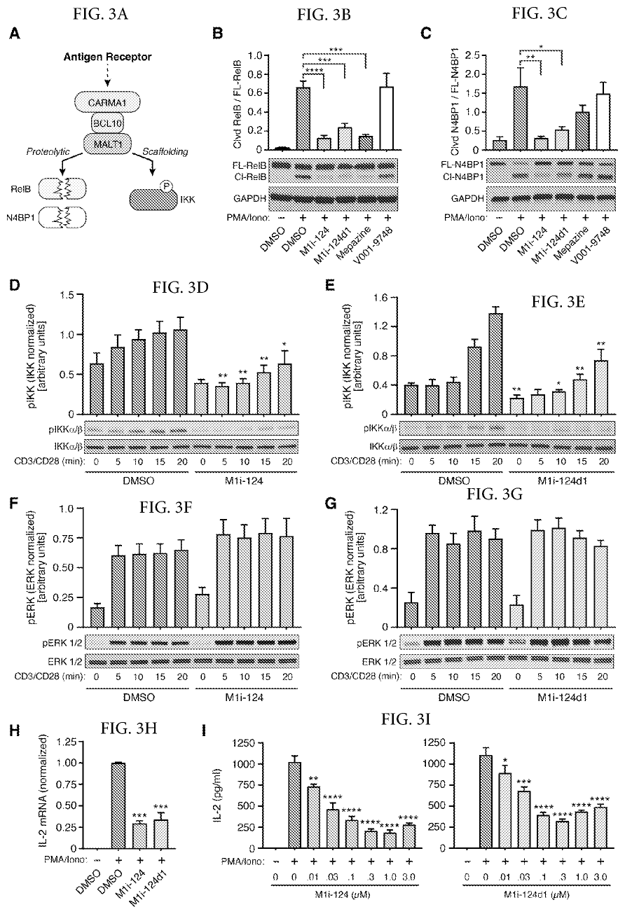

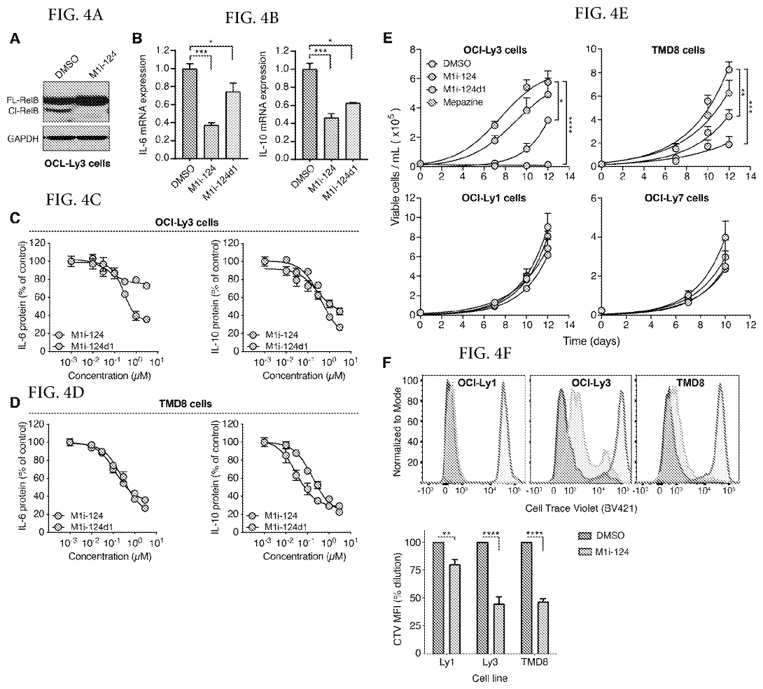

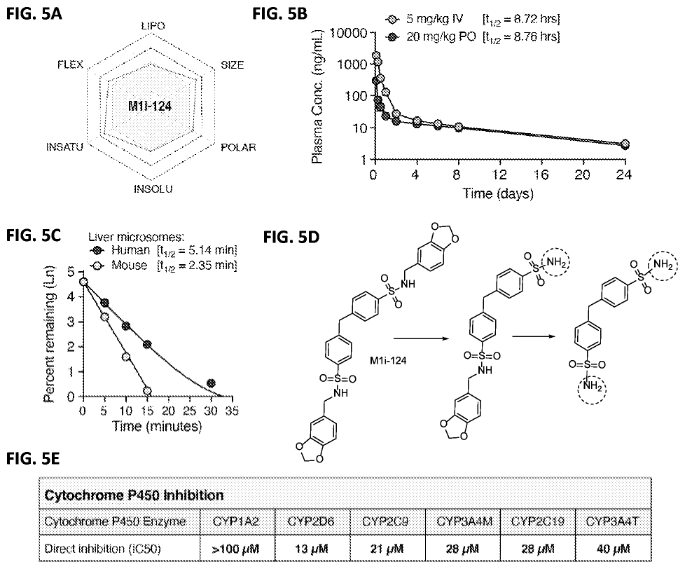

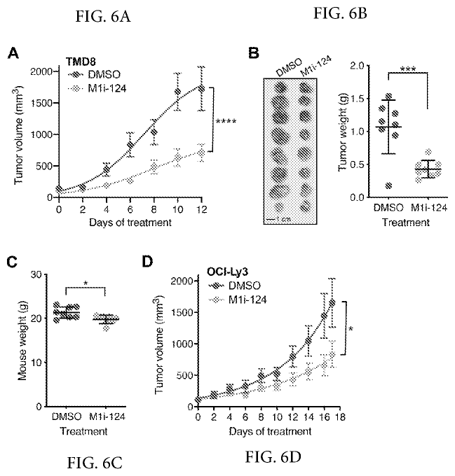

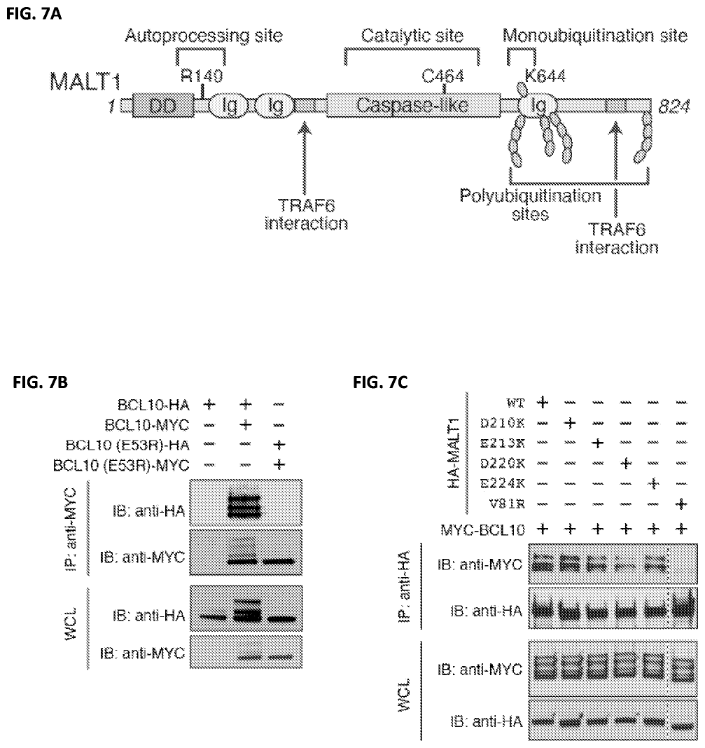

### DETAILED DESCRIPTION

### Overview

Disclosed herein are small molecule inhibitors that block the interaction between B-cell lymphoma 10 protein (BCL10) and mucosa-associated lymphoid tissue lymphoma translocation protein 1 (MALT1), thereby inhibiting both the protease and scaffolding activities of MALT1, and MALT1-dependent downstream signaling, including IL-6 and IL-10 secretion by B-cell lymphoma cells and IL-2 transcription and secretion by Jurkat T cells. As these compounds abrogate all MALT1 activity (protease and scaffolding activities) they may be significantly more efficacious and wide ranging in terms of therapeutic applications for MALT1 driven malignancies and overactive inflammatory diseases.

MALT1 inhibitors previously proposed for treatment of cancers or inflammation are protease inhibitors and thus prevent maximal NF-κB activation in disease contexts where it is overactive (e.g. ABC-DLBCL). MALT1 protease inhibitors aggravate the problem of autoimmune activation as has been observed in mice. The compounds disclosed herein inhibit activation of MALT1 by blocking its interaction with BCL10, rather than targeting only the protease function of MALT1, thus affecting the various components of the T & B cell immune responses.

Terminology The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

The term “alkoxy” refers to a straight, branched or cyclic hydrocarbon configuration and combinations thereof, including from 1 to 20 carbon atoms, preferably from 1 to 8 carbon atoms (referred to as a “lower alkoxy”), more preferably from 1 to 4 carbon atoms, that include an oxygen atom at the point of attachment. An example of an “alkoxy group” is represented by the formula —OR, where R can be an alkyl group, optionally substituted with an alkenyl, alkynyl, aryl, aralkyl, cycloalkyl, halogenated alkyl, alkoxy or heterocycloalkyl group. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1- hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1- hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Alkynyl” refers to a cyclic, branched or straight chain group containing only carbon and hydrogen, and unless otherwise mentioned typically contains one to twelve carbon atoms, and contains one or more triple bonds. Alkynyl groups may be unsubstituted or substituted. “Lower alkynyl” groups are those that contain one to six carbon atoms.

An “animal” refers to living multi-cellular vertebrate organisms, a category that includes, for example, mammals and birds. The term mammal includes both human and non-human mammals. Illustrative non-human mammals include animal models (such as mice), non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl). The term “aralkyl” refers to an alkyl group wherein an aryl group is substituted for a hydrogen of the alkyl group. An example of an aralkyl group is a benzyl group.

“Aryl” refers to a monovalent unsaturated aromatic carbocyclic group having a single ring (e.g., phenyl) or multiple condensed rings (e.g., naphthyl or anthryl), which can optionally be unsubstituted or substituted. A “heteroaryl group,” is defined as an aromatic group that has at least one heteroatom incorporated within the ring of the aromatic group. Examples of heteroatoms include, but are not limited to, nitrogen, oxygen, sulfur, and phosphorous. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The aryl or heteroaryl group can be substituted with one or more groups including, but not limited to, alkyl, alkynyl, alkenyl, aryl, halogen, nitro, amino, ester, ketone, aldehyde, hydroxy, carboxylic acid, or alkoxy, or the aryl or heteroaryl group can be unsubstituted.

“Bicyclic” or “bicyclyl”, as used here, in refers to a ring assembly of two rings where the two rings are fused together. The rings may be a carbocyclyl, a heterocyclyl, an unsaturated aromatic group or a mixture thereof.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

“Halo” or “halogen”, as used herein, refers to fluoro, chloro, bromo, and iodo. The term “heterocyclic” refers to a closed-ring compound, or radical thereof as a substituent bonded to another group, particularly other organic groups, where at least one atom in the ring structure is other than carbon, and typically is oxygen, sulfur and/or nitrogen.

“Inhibiting” refers to inhibiting the full development of a disease or condition. “Inhibiting” also refers to any quantitative or qualitative reduction in biological activity or binding, relative to a control.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl). “Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. A “prophylactic” treatment is a treatment administered to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono—, di—, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound. The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs may include compounds having a phosphonate, hydroxy, thio and/or amino group functionalized with any group that is cleaved in vivo to yield the corresponding amino, hydroxy, thio and/or phosphonate group, respectively. Examples of prodrugs can include, without limitation, compounds having an acylated amino group and/or a phosphonate ester or phosphonate amide group.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus the compounds described can exist in different stereoisomeric forms. Accordingly, compounds and compositions may be provided as individual pure enantiomers or as stereoisomeric mixtures, including racemic mixtures. In certain embodiments the compounds disclosed herein may be synthesized in or may be purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center (C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc.

### Compounds

In certain embodiments, the MALT1 inhibitor is a compound, or a pharmaceutically acceptable salt thereof, having a structure of:

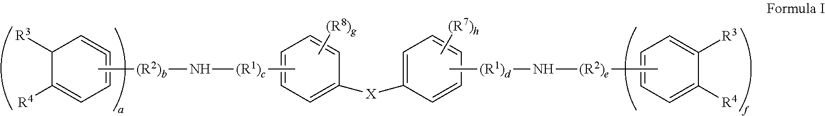

wherein X is (—CH₂—)_{y}, (—CH(R¹³)—)_{y}, (—C(R¹⁴)(R¹⁵)—)_{y}, —O—, —S(O₂)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH(R)¹⁰)ₓ, (—C(R¹¹)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d or e is 1; and
provided that if —(R²)_{b}— is H, then a is 0, and if —(R²)ₑ— is H, then f is 0.

In certain embodiments, the

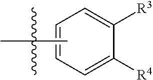

group(s) of formula I is

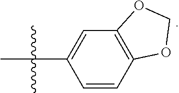 [SMILES: *c1ccc2c(c1)OCO2]

In certain embodiments, the

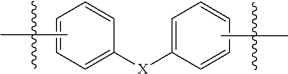

group of formula I is

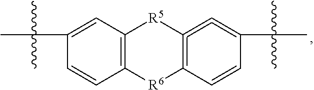

independently —CH₂—, —O—or N.
wherein R⁵ and R⁶ are each

In certain embodiments, at least one of R¹ or R² is —S(O)₂—.

In certain embodiments, at least one of R¹ or R² is (—CH₂—)ₓ, wherein x is 1.

In certain embodiments, X is —CH₂—.

In certain embodiments, —(R¹)_{c}— is —S(O)₂—, and —(R¹)ₑ— is —S(O)₂—.

In certain embodiments, —(R²)_{b}— is —CH₂—, —(R¹)_{c}— is —S(O)₂—, X is —CH₂—, —(R¹)_{d}— is —S(O)₂—, and —(R²)ₑ— is —CH₂—.

In certain embodiments, at least one of R³ and R⁴ is alkoxy.

In certain embodiments, each of a, b, c, d, e, and f is 1.

In certain embodiments, each of b, c, d, and e is 1.

In certain embodiments, at least one of a or f is 0.

In certain embodiments, —(R²)ₑ— is H.

In certain embodiments, —(R²)_{b}— is H and —(R²)ₑ— is H.

In certain embodiments, —(R²)ₑ— is H and —(R¹)_{d}— is —S(O)₂.

In certain embodiments, —(R²)_{b}— is H, —(R²)ₑ— is H, —(R⁴)_{c}— is —S(O)₂—, and —(R¹)_{d}— is —S(O)₂.

In certain embodiments, R³ and R⁴ together form a fused bicyclic structure with the benzene ring that is adjacent to R³ and R⁴, and the fused bicyclic structure includes at least one heteroatom.

In certain embodiments, the compound is:

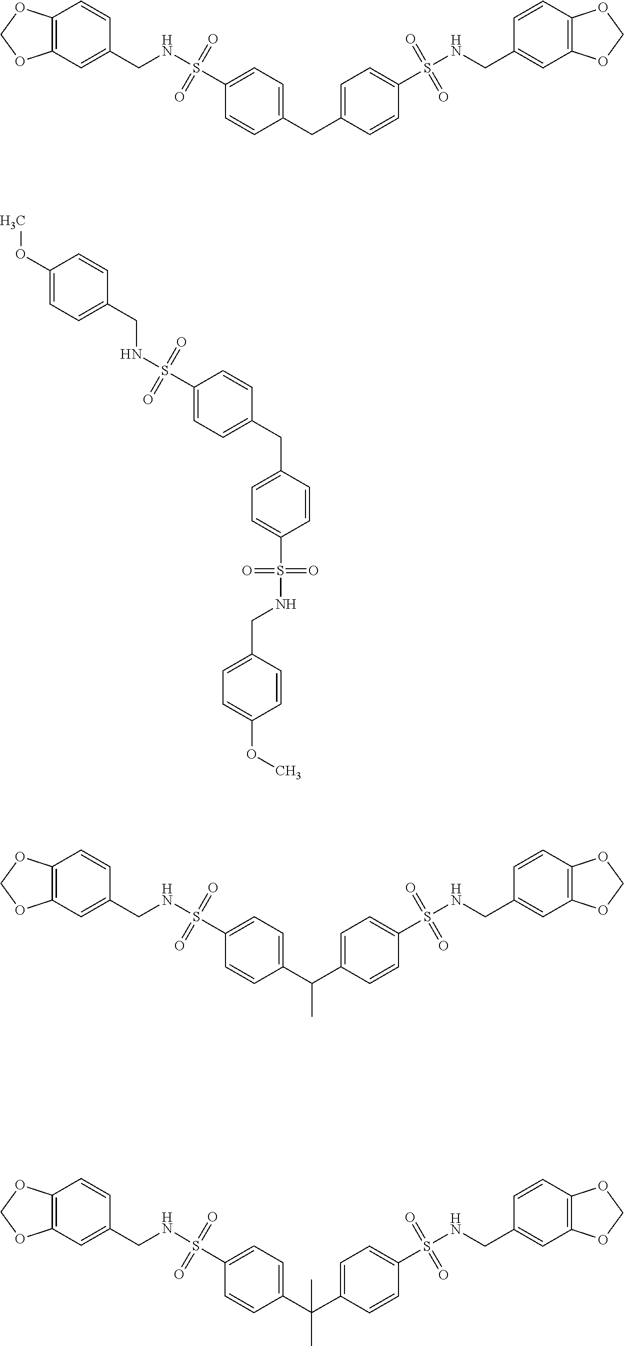 [SMILES: O=S(=O)(NCc1ccc2c(c1)OCO2)c1ccc(Cc2ccc(S(=O)(=O)NCc3ccc4c(c3)OCO4)cc2)cc1 ; COc1ccc(CNS(=O)(=O)c2ccc(Cc3ccc(S(=O)(=O)NCc4ccc(OC)cc4)cc3)cc2)cc1 ; CC(c1ccc(S(=O)(=O)NCc2ccc3c(c2)OCO3)cc1)c1ccc(S(=O)(=O)NCc2ccc3c(c2)OCO3)cc1 ; CC(C)(c1ccc(S(=O)(=O)NCc2ccc3c(c2)OCO3)cc1)c1ccc(S(=O)(=O)NCc2ccc3c(c2)OCO3)cc1]

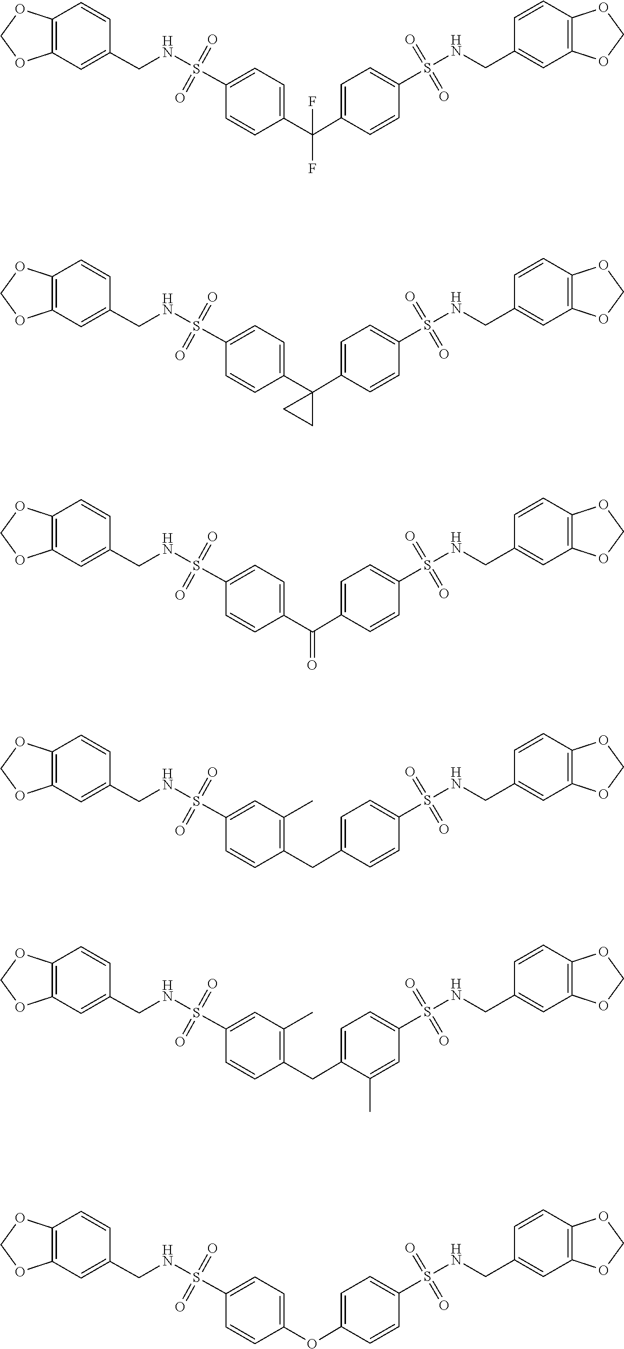 [SMILES: O=S(=O)(NCc1ccc2c(c1)OCO2)c1ccc(C(F)(F)c2ccc(S(=O)(=O)NCc3ccc4c(c3)OCO4)cc2)cc1 ; O=S(=O)(NCc1ccc2c(c1)OCO2)c1ccc(C2(c3ccc(S(=O)(=O)NCc4ccc5c(c4)OCO5)cc3)CC2)cc1 ; O=C(c1ccc(S(=O)(=O)NCc2ccc3c(c2)OCO3)cc1)c1ccc(S(=O)(=O)NCc2ccc3c(c2)OCO3)cc1 ; Cc1cc(S(=O)(=O)NCc2ccc3c(c2)OCO3)ccc1Cc1ccc(S(=O)(=O)NCc2ccc3c(c2)OCO3)cc1 ; Cc1cc(S(=O)(=O)NCc2ccc3c(c2)OCO3)ccc1Cc1ccc(S(=O)(=O)NCc2ccc3c(c2)OCO3)cc1C ; O=S(=O)(NCc1ccc2c(c1)OCO2)c1ccc(Oc2ccc(S(=O)(=O)NCc3ccc4c(c3)OCO4)cc2)cc1]

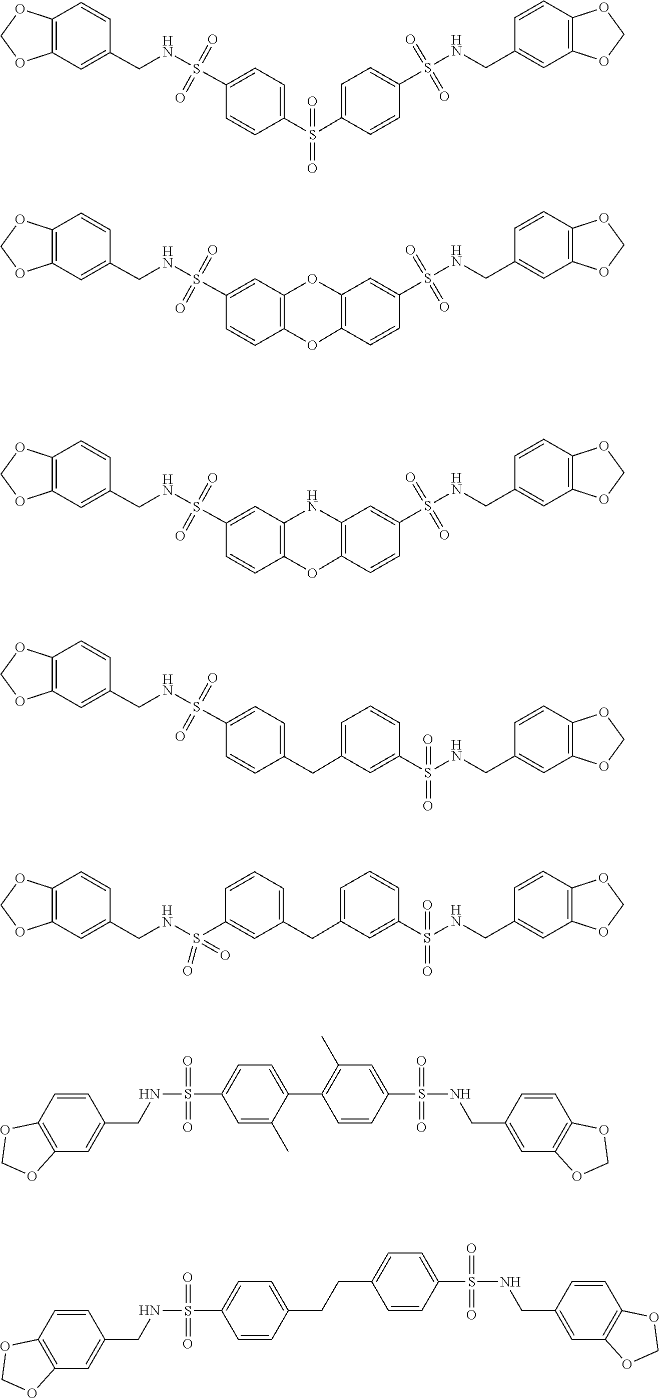

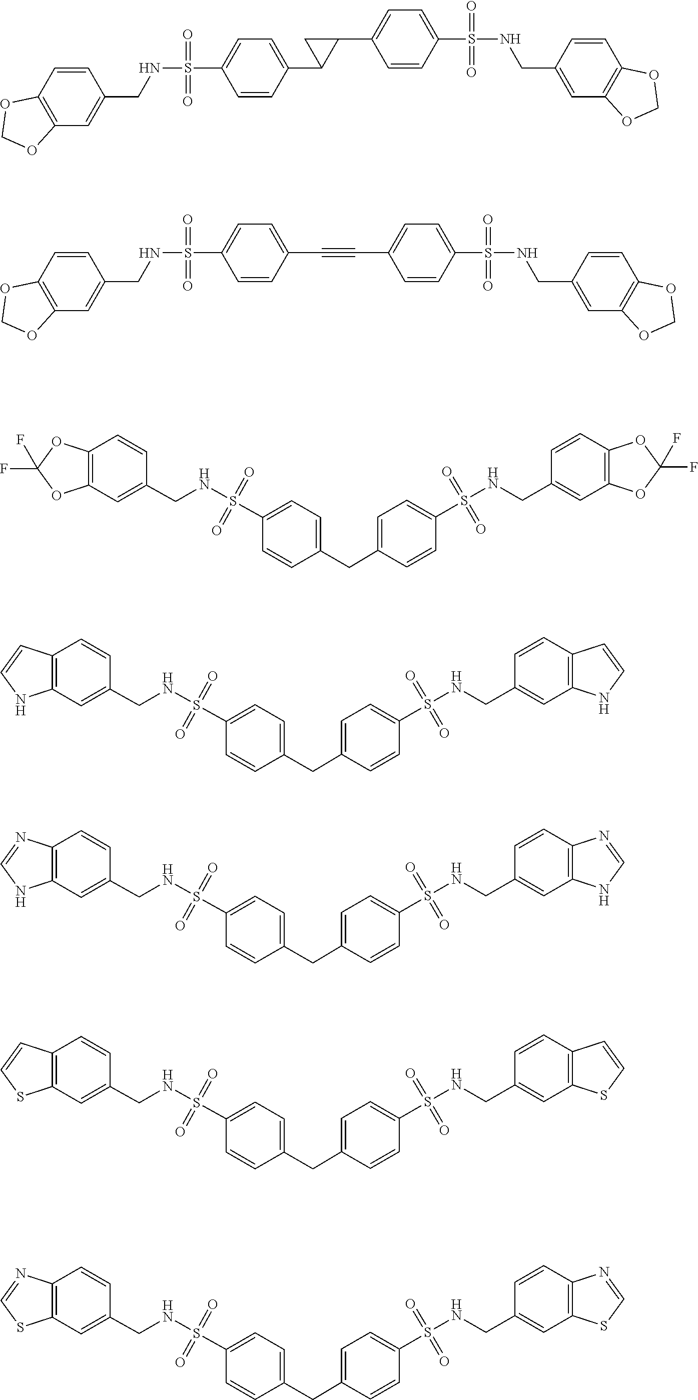

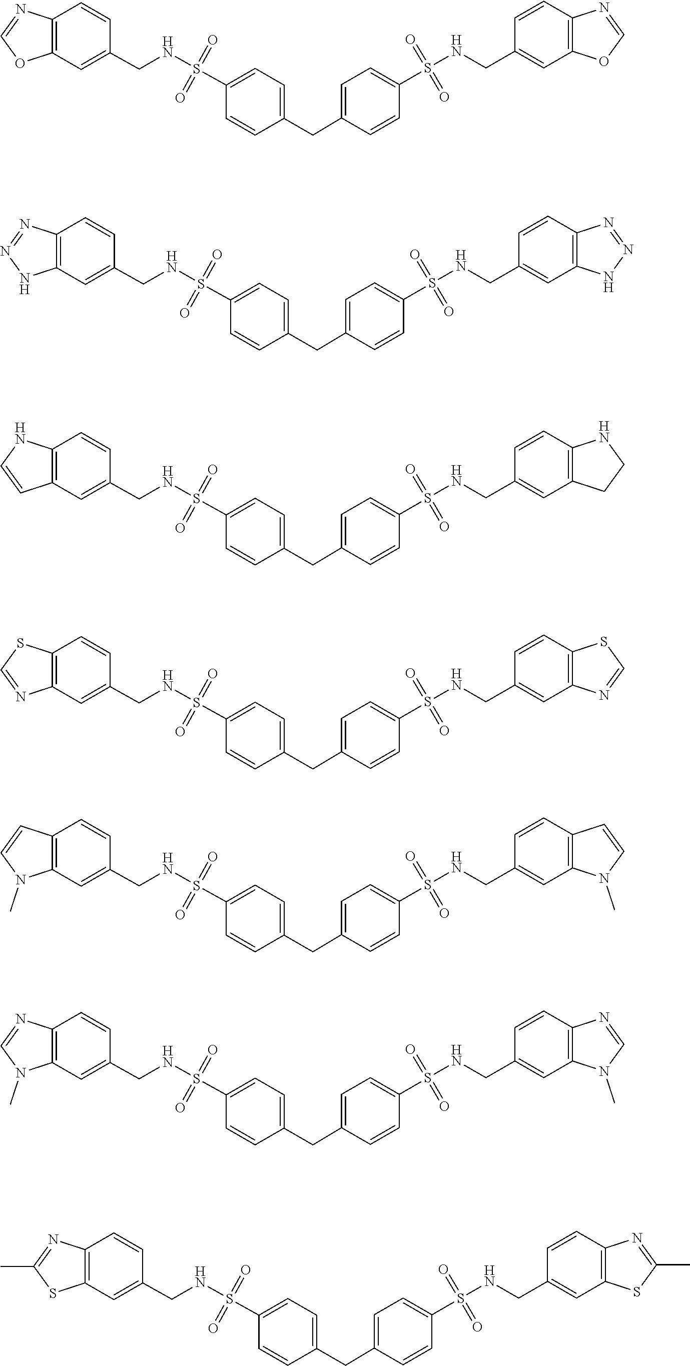

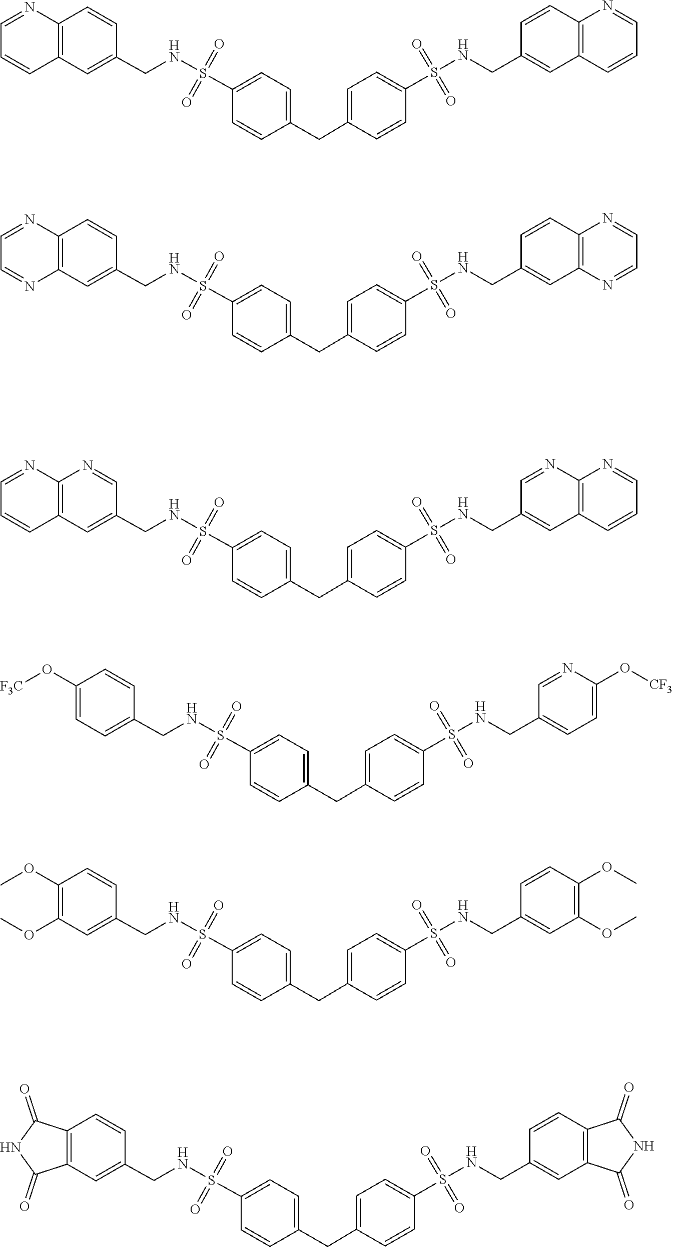

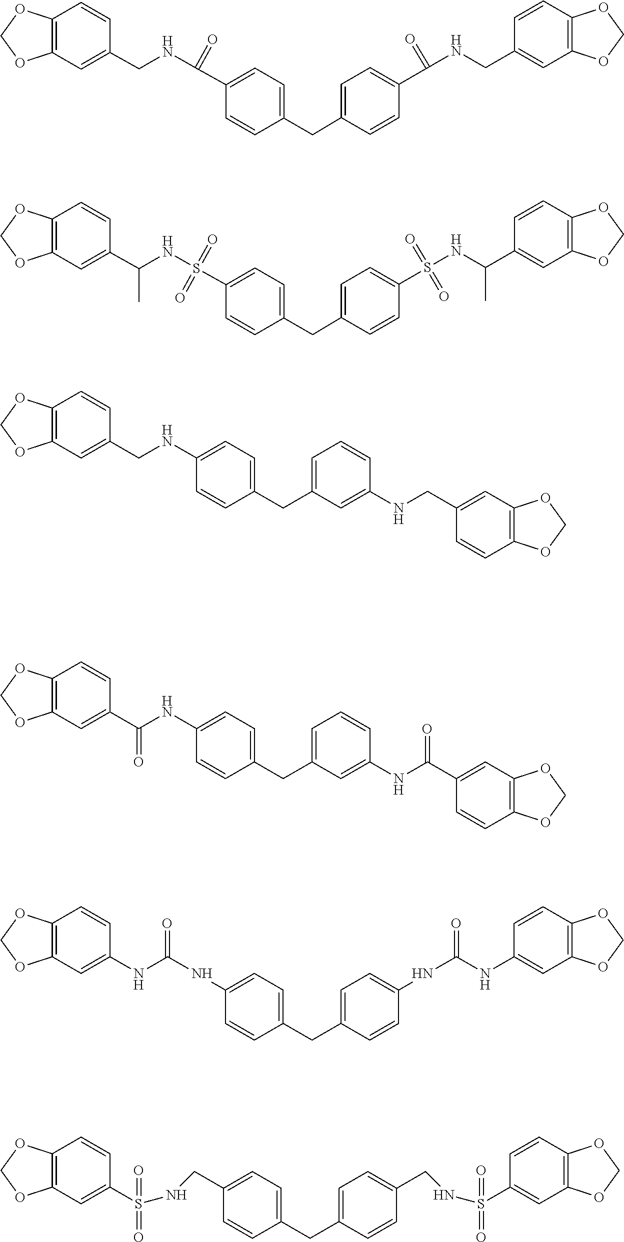 [SMILES: O=C(NCc1ccc2c(c1)OCO2)c1ccc(Cc2ccc(C(=O)NCc3ccc4c(c3)OCO4)cc2)cc1 ; CC(NS(=O)(=O)c1ccc(Cc2ccc(S(=O)(=O)NC(C)c3ccc4c(c3)OCO4)cc2)cc1)c1ccc2c(c1)OCO2 ; c1cc(Cc2ccc(NCc3ccc4c(c3)OCO4)cc2)cc(NCc2ccc3c(c2)OCO3)c1 ; O=C(Nc1ccc(Cc2cccc(NC(=O)c3ccc4c(c3)OCO4)c2)cc1)c1ccc2c(c1)OCO2 ; O=C(Nc1ccc(Cc2ccc(NC(=O)Nc3ccc4c(c3)OCO4)cc2)cc1)Nc1ccc2c(c1)OCO2 ; O=S(=O)(NCc1ccc(Cc2ccc(CNS(=O)(=O)c3ccc4c(c3)OCO4)cc2)cc1)c1ccc2c(c1)OCO2]

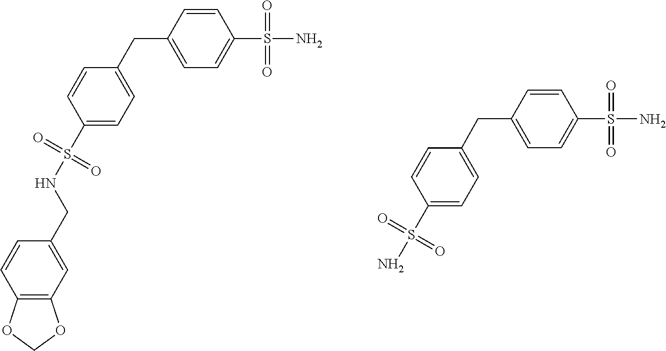 [SMILES: NS(=O)(=O)c1ccc(Cc2ccc(S(=O)(=O)NCc3ccc4c(c3)OCO4)cc2)cc1 ; NS(=O)(=O)c1ccc(Cc2ccc(S(N)(=O)=O)cc2)cc1]

An illustrative scheme for making compounds of formula I is shown below:

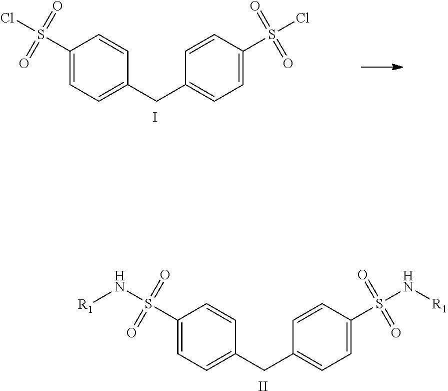

Compound I is reacted with an amine (R₁—NH₂) and reflux in ethanol to generate compound II. In this scheme the R¹ group is the same as

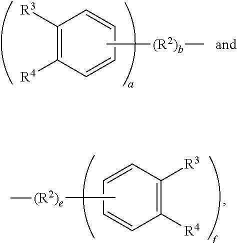

respectively.

In an independent embodiment, the MALT1 inhibitor is a compound having a structure of:

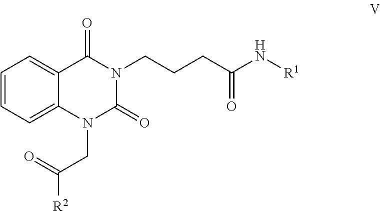

wherein R¹ and R² are each independently aryl, substituted aryl, aralkyl, or substituted aralkyl. In certain embodiments, R¹ and R² are each independently phenyl, substituted phenyl (e.g., halo-substituted phenyl or alkoxy-substituted phenyl), benzyl, or substituted benzyl (e.g., —(CH₂)_{z}—Ar, wherein z is 1 to 4, and Ar is substituted phenyl (e.g., alkoxy-substituted phenyl or halo-substituted phenyl)).

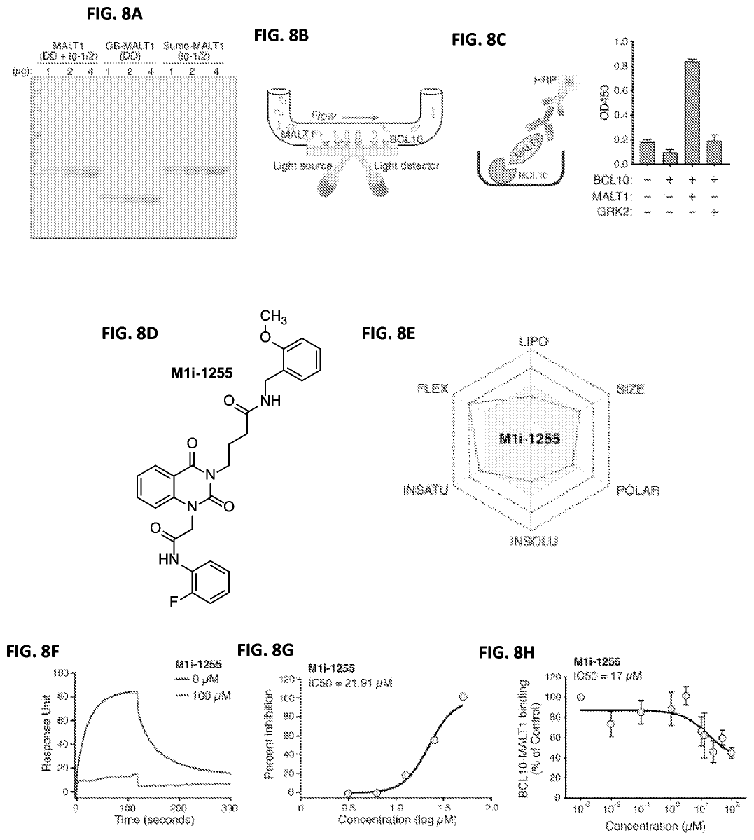

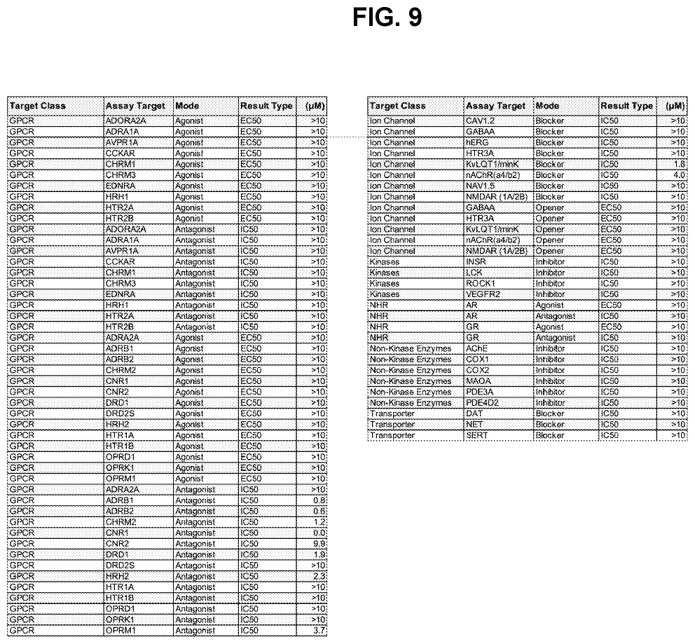

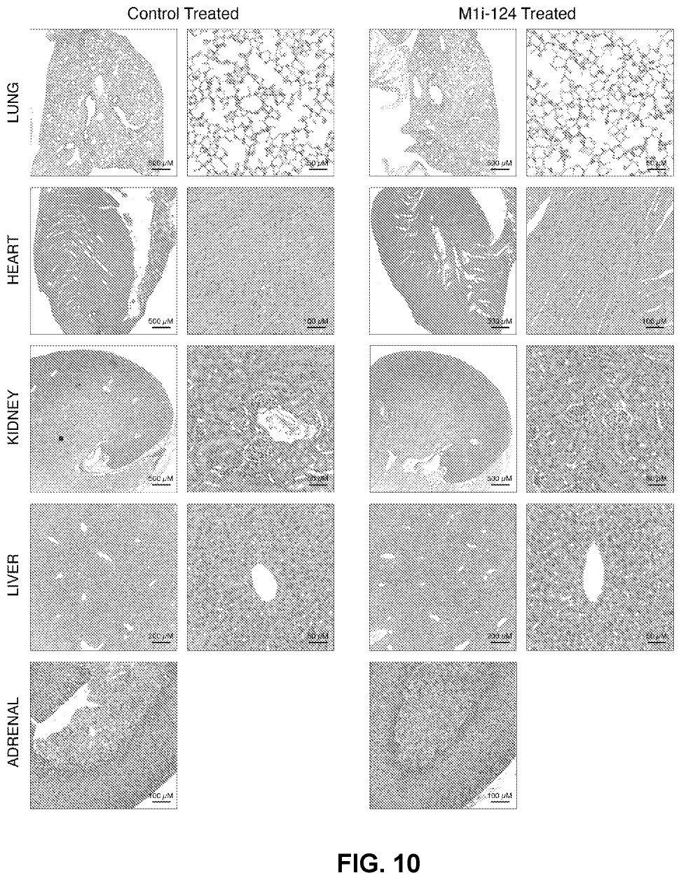

An example of a compound of formula V is shown in FIG. 8D.

An illustrative scheme for making compounds of formula V is shown below:

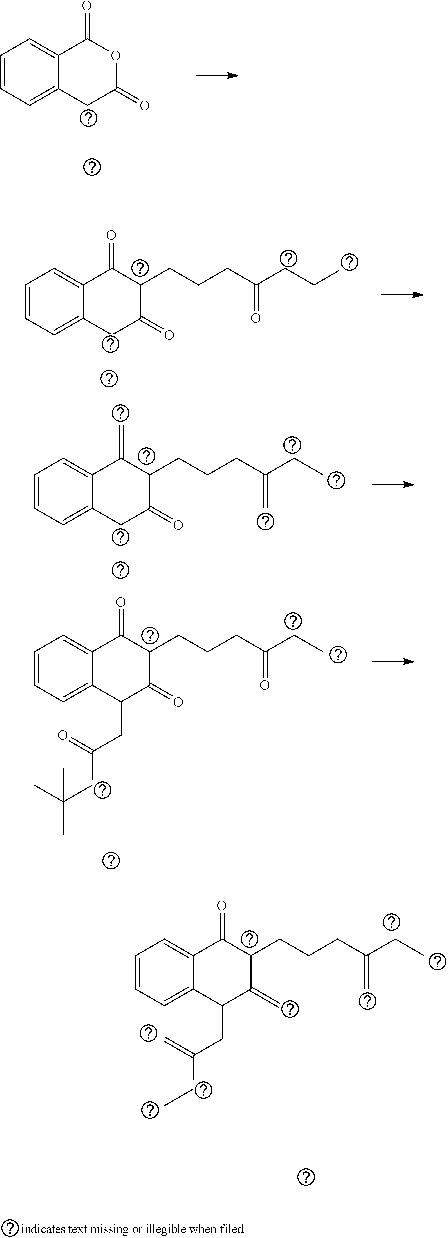 [SMILES: O=C1Cc2ccccc2C(=O)O1 ; CCCC(=O)CCCC1C(=O)Cc2ccccc2C1=O ; C=C(CC)CCCC1C(=C)c2ccccc2CC1=O ; CCC(=O)CCCC1C(=O)c2ccccc2C(CC(=O)CC(C)(C)C)C1=O ; C=C(CC)CCCC1C(=C)C(CC(=C)CC)c2ccccc2C1=O]

Compound I is reacted with an amine and subsequently cyclized by phosgene or its equivalents such as triphosgene to provide compound II. Compound II is hydrolyzed to the carboxylic acid and coupled with an amine such as benzylamine or cyclopentylamine to give compound III. Compound III is alkylated with an acetic acid derivative, such as bromo-acetic acid methyl ester, to provide compound IV. Compound IV is hydrolyzed, and the resulting carboxylic acid coupled with an amine, e.g., aryl amine, to provide compound V.

### Method of Use and Pharmaceutical Compositions

The compounds disclosed herein are useful as MALT1 inhibitors. In certain embodiments, the compounds may be administered to a subject in need of, or has been recognized as being in need of, treatment with a MALT1 inhibitor. These new class of protein-protein interaction inhibitors provide balanced inhibition of MALT1, avoiding the potential adverse immunomodulatory actions (induced autoimmunity) that result from inhibiting only one arm of MALT1 action, as is seen with the currently available MALT1 protease inhibitors.

The compounds disclosed herein may be administered to a subject having, suspected of having, or at risk of having, a T- and B-cell malignancy, a carcinoma, an autoimmune disease, an inflammatory disease, or an allergy.

Illustrative T- and B-cell malignancies include lymphoma, particularly lymphomas with mutations that result in deregulated MALT1 activity including Activated B Cell subtype of Diffuse Large B Cell Lymphoma (ABC-DLBCL), Mantle Cell Lymphoma (MCL), Chronic Lymphocytic Leukemia (CLL), Acute T-cell Leukemia/Lymphoma (ATLL), Cutaneous T-cell Lymphoma (CTCL), Sezary Syndrome, and peripheral T-cell Lymphoma (PTCL).

Certain types of leukemia is another disease category in which MALT1 function is deregulated.

Illustrative carcinomas include breast cancer, pancreatic cancer, melanoma, and glioblastoma.

Illustrative autoimmune and inflammatory diseases include, but are not limited to, psoriasis, multiple sclerosis, rheumatoid arthritis, Sjogren's syndrome, colitis, systemic lupus erythematosus, graft-versus-host disease (GVHD), and asthma.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent (such as an anti-retroviral agent) for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 10 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### EXAMPLES

### Materials and Methods

### Cell Lines and Cell Culture

The DLBCL cell lines, OCI-Ly1, OCI-Ly3, and OCI-Ly7, were provided by Dr. Mark Minden (University Health Network, Toronto, Ontario, Canada). TMD8 cells were provided by Dr. Louis Staudt (NCI, NIH). OCI-Ly1 and OCI-Ly7 cells were cultured in Iscove's Modified Dulbecco's Medium (IMDM) supplemented with 10% FBS. OCI-Ly3 cells were cultured in IMDM supplemented with 20% FBS. TMD8 and Jurkat T cells were cultured in RPMI1640 medium with 10% FBS. HEK293T cells were cultured in DMEM medium with 10% FBS. All media were supplemented with 100 U/ml penicillin streptomycin. Stimulation of Jurkat cells was conducted by adding 5 ng/ml of phorbol 12-myristatel3 acetate (PMA)(Sigma-Aldrich) and 1μM ionomycin (Calbiochem), or by adding 5-10 μL/mL of ImmunoCult Human CD3/CD28 (STEMCELL Technologies).

### Antibodies

Rabbit anti-phospho-IKKa/b antibody (Cell Signaling #2078)

Rabbit anti-IKKa/b antibody (H-470)(Santa Cruz #sc-7607)

Rabbit anti-phospho-p44/42 MAPK (Erk1/2) antibody (Cell Signaling #9101)

Rabbit anti-P44/42 MAPK (Erk1/2) antibody (Cell Signaling #9102)

Rabbit anti-RelB antibody (C1E4)(Cell Signaling #4922)

Rabbit anti-N4BP1 antibody (Cohesion Biosciences #CPA2415)

Mouse anti-GAPDH antibody (6C5)(Santa Cruz #sc-32233, Lot #H0411)

Rabbit anti-MALT1 antibody (Cell Signaling #2494)

Mouse anti-Bc110 antibody (331.3)(Santa Cruz Cat #sc-5273)

Rabbit anti-Bc110 antibody (H-197)(Santa Cruz Cat #sc-5611)

Chemicals and other reagents:

Cell Trace Violet (ThermoFisher Scientific Cat #C34557); Sytox Blue (ThermoFisher Scientific Cat #S34857); Annexin V 488 (ThermoFisher Scientific Cat #A13201); Propidium Iodide (ThermoFisher Scientific Cat #P3566); Annexin Binding buffer (5×)(ThermoFisher Scientific Cat #V13246); Lipofectamine 3000 (ThermoFisher Scientific Cat #L3000001); Proteasome inhibitor MG132 (Calbiochem); phorbol 12-myristate13 acetate (PMA)(Sigma-Aldrich); ionomycin (Calbiochem); ImmunoCult Human CD3/CD28 (STEMCELL Technologies); high-binding 96-well ELISA microplate (Greiner Cat #655061); ECL plus (Pierce PI80196X3)

### Plasmids

pM3-VP16 vector (Takara Cat #630305); pcDNA3-based plasmids encoding Myc- or HA-tagged proteins (RICK, BCL10, MAVS, or deletion mutants thereof), have been described previously (1, 2). Myc-BCL10 E53R, HA-BCL10 E53R, HA-Ig1-2 V189R, HA-Ig1-2 F187A, HA-MALT1 V81R, HA-MALT1 D210K, HA-MALT1 E213K, HA-MALT1 D220K, HA-MALT1 E224K, HA-MALT1 D210K/E213K, and HA-MALT1 D220K/E224K were made from the corresponding pcDNA3 wild-type constructs using the QuikChange II site-directed mutagenesis kit (Agilent, Cat #200523); HA-Ig1-2 (MALT1 AA 128-330), HA-Ig1-2 W203_Q205delinsAAA, HA-Ig1-2 P254_P256delinsAAA HA-Ig1-2 H257_Q259delinsAAA, HA-Ig1-2 H269_T271delinsAAA, HA-Ig1-2 K272_L274, HA-MALT1, and HA-MALT1 V189R were constructed by Vector Builder.

TaqMan RT-PCR probe sets

IL2: Hs00174114_m1

CXCL8: Hs00174103_m1

IL-10: Hs0091622_m1

IL-6: Hs00174131_m1

GAPDH: Hs02786624_g1

### Purified Proteins

His-tagged recombinant human full-length Bc110 protein (ProteinTech Cat #Ag12162); GST-tagged recombinant human full-length Bcl10 protein (Abnova Cat #H00008915-P01); GST-tagged recombinant human full-length MALT1 protein (Novus Biologicals Cat #H00010892-P01); MALT protein fragments (DD +Ig1/2, GB-tagged MALT1 DD; Sumo-tagged MALT1 Ig-1/2) were produced and purified by column chromatography in the laboratory of Dr. Zaneta Nikolovska-Coleska).

### Kits

Luciferase Reporter Assay system (Promega Cat #E1501); (3-galactosidase enzyme assay system (Promega Cat #E2000); RNeasy Plus Mini Kit (Qiagen Cat #74136); QuikChange II site-directed mutagenesis kit (Agilent, Cat #200523); High-Capacity cDNA Reverse Transcription Kit (Applied Biosystems Cat #4368814); Human IL-2 ELISA (BioLegend Cat #431804); Human IL-6 ELISA (BioLegend Cat #430504); Human IL-10 ELISA (BioLegend Cat #430604)

### 3D Molecule Visualizations

PyMOL (Schrodinger) was used to generate 3D renderings of the MALT1 Ig 1/2 domain using the crystal structure deposited in the RCSB Protein Data Bank (PDB identifier: 3K0W)

### In Silico Small Molecule Screen

The LibDock program from Discovery Studio 3.5 was used to perform a structure-guided in silico screen of 3 million compounds to identify candidate molecules that could potentially fit within the identified groove of MALT1 Igl-Ig2 domain, found to be critical for BCL10-MALT1 interaction. Lipinski's rule of five filters were applied to enrich for compounds with drug-like properties. Top hits were further evaluated for their potential to disrupt the BCL10-MALT1 interaction through secondary SPR and ELISA-based assays as described below.

### Surface Plasmon Resonance (SPR)

SPR is a real-time, label-free optical technique to detect biomolecular interactions. All SPR experiments were performed on a Biacore 2000. Full-length Bcl10 molecules (Abnova Cat #H00008915-P01) were immobilized on a gold sensor chip surface and analyte solution containing MALT1 DD+Ig1-Ig2, MALT1 Ig1-Ig2, or MALT1 DD protein was passed over the surface through a series of flow cells. During the course of the interaction, polarized light is directed toward the sensor surface and the angle of minimum intensity reflected light is detected. This angle changes as molecules bind and dissociate and the interaction profile is recorded in real time in a sensorgram. The binding kinetic parameters were obtained using the Langmuir model (3) for global fitting 1:1 binding ratio where one ligand molecule interacts with one analyte molecule. To verify the binding constants, additional local fitting was performed.

### ELISA-Based Protein-Protein Interaction Assay

On a high-binding 96-well ELISA microplate (Greiner Cat #655061), 100 ng of His-tagged recombinant human full-length Bcl10 protein (ProteinTech Cat #Ag12162) in 100 ul PBS was allowed to adhere overnight in 4° C. The plate was blocked with 5% BSA in PBS for 1 hour at room temperature. After washing twice with PBS-T, a mixture of 100 ng of GST-tagged recombinant human full-length MALT1 protein (Novus Biologicals Cat #H00010892-P01), +/−compound that was preincubated for 1 hour in 100 ul PBS, was added to the plate for 1 hour at room temperature. After washing three times with PBS-T, anti-MALT1 antibody in 5% BSA in PBS was added at 1:1000 dilution for 1 hour at room temperature. After washing three times with PBS-T, HRP-conjugated secondary antibody in 5% BSA in PBS was added at 1:5000 dilution for 1 hour at room temperature. After washing three times with PBS-T, 100 ul of TMB was added for 5 minutes, and 100 ul of 2 M sulfuric acid was added to stop the reaction. The plate was read at 450 nm using a spectrophotometer (Molecular Devices SpectraMax i3).

### Immunoprecipitation

For immunoprecipitation, cells were rinsed in PBS and lysed in NP40 lysis buffer (150 mM NaCl, 2 mM EDTA, 10% glycerol, 1% Nonidet P-40, 20 mM Tris Hcl, pH 7.4) containing protease and phosphatase inhibitors. Lysates were cleared and supernatants containing ˜0.5 mg of protein were incubated overnight at 4° C. with c-myc monoclonal antibody-agarose beads (Takara) Beads were pelleted at 1000 rpm for 2 min and washed a minimum of three times with NP-40 buffer containing protease inhibitors. To elute proteins from beads, 2× Laemmli sample buffer (Promega) was added and samples were boiled prior to SDS gel electrophoresis. Proteins were transferred to nitrocellulose via semi-dry or tank transfer and membranes were probed with anti-HA-peroxidase antibody (Roche). Membranes were stripped with Restore™ stripping blot buffer (Thermo Scientific) and reprobed with rabbit anti-myc (Cell signaling) followed by horseradish peroxidase conjugated anti-rabbit IgG (Promega). Whole cell lysates were also analyzed via Western blot analysis and probed as above.

### RelB and N4BP1 Cleavage Assays

In the Jurkat cell model, cells were treated with 1μM PPIi compounds for 20 hours, followed by the addition of 5 μM MG132 for 1 additional hour, prior to stimulation with 5 μl/ml anti-CD3/CD28 for 1 hour. In the DLBCL model, cells were treated overnight with 1μM PPIi compounds prior to the addition of 5 μM MG132 for 4 hours. Following treatments, cells were pelleted, rinsed twice with cold PBS, and lysed with 1× Millipore RIPA buffer containing protease inhibitors. Lysates were subjected to SDS-PAGE on 8% Bolt gels, and proteins transferred to nitrocellulose. Membranes were blocked with 5% non-fat dry milk and probed with anti-RelB or anti-N4BP1 antibody, followed by anti-rabbit secondary antibody at 1:500 dilution (Fisher scientific PRW4011). Blots were also probed for GAPDH as a loading control. Blots were developed using ECL plus (Pierce PI80196X3) chemiluminescent detection. Band quantification was performed with ImageJ in order to calculate the ratio of cleaved to full-length RelB and N4BP1 protein.

### Quantitative RT-PCR

Total RNA extraction was performed using RNeasy Plus Mini Kit (Qiagen Cat #74136). 1 ng RNA was transcribed into cDNA using High-Capacity cDNA Reverse Transcription Kit (Applied Biosystems Cat #4368814) according to manufacturer's instructions. RT-PCR reactions were performed using TaqMan gene expression probe sets.

### ELISA

Expression of cytokines from cell culture supernatants was measured 24h after treatment with 0, 0.01, 0.03, 0.1, 0.3, 1, or 3 uM of compounds using the following ELISA kits according to the manufacturer's instructions: Human IL-2 ELISA (BioLegend Cat #431804), Human IL-6 ELISA (BioLegend Cat #430504), and Human IL-10 ELISA (BioLegend Cat #430604).

### Cell Viability and Proliferation

Cell number was counted using the trypan blue dye exclusion method with Vi-CELL cell viability analyzer (Beckman Coulter) at days 7, 10, and 12 of treatment with compounds. Cell proliferation was specifically evaluated using the CellTrace Violet reagent (ThermoFisher Cat #C34571) which undergoes dilution with each round of cell division. 1.5×10⁶ cells were washed twice in PBS and resuspended in 2 mls of PBS.

CellTrace Violet was added at a final concentration of 5 μM and the cells were incubated at 37° C. in the dark. At the end of incubation, 5% FBS media was added and cells were centrifuged (400×g, 5 min) and resuspended in fresh 5% media. 100,000 cells were plated in 4 ml of media/well (6 well plate) with either 1 μl/ml DMSO vehicle control or 1 μM final concentration of PPIi compound. After 10 minutes, extra labeled cells and unlabeled cells were analyzed by flow cytometry on a BD Biosciences LSR II cytometer to obtain appropriate baseline settings. At day 6, the cells were stained with a live/dead cell dye (PI) and evaluated by flow cytometry using the baseline settings and FlowJo software. Cell trace violet was detected in the BV421 channel.

### Xenograft Models of DLBCL

Tumors were engrafted into 8-week old female SCID NOD.CB17-Prkdc^{scid}/J (nonobese diabetic/severe combined immunodeficiency) mice from Jackson Laboratory by subcutaneous injection of either 10×10⁶ TMD8 cells or OCI-Ly3 cells, resuspended in Matrigel (Cultrex Basement Membrane Extract, type 3, Pathclear; Trevigen) into the right flank of individual mice, with 10 mice for DMSO vehicle control and 10 mice for compound M1i-124. After visual appearance of tumor (i.e. when tumors reach an average volume of 100 mm 3), mice were randomized to M1i-124 or DMSO vehicle control groups, and received once daily intraperitoneal injection of 50 mg/kg of M1i-124 (corresponding to 42 μl for a 25g mouse) or equivalent volume of DMSO for up to 12 days. Tumor size was measured every other day using a caliper and calculated using the formula: (smallest diameter 2× largest diameter)/2. Mice were sacrificed 24 hours after the 12^{th} injection in the case of TMD8 xenografts or after the 18^{th} injection in the case of OCI-Ly3 xenografts. Tumors and various organs, including lung, heart, liver, and kidney were collected for histological analyses. All experimental procedures involving animals have been approved by University of Pittsburgh IACUC.

### Pharmacokinetics, Toxicity, and ADME Studies

Studies were performed using the services of Touchstone Biosciences and Eurofins Pharma Discovery Services. To determine in vivo plasma half-life of M1i-124, male CD-1 mice were fasted overnight before being dosed with 5 mg/kg M1i-124 via intravenous bolus injection or with 20 mg/kg via oral gavage. All blood samples (30-50 ul per sample) were taken via tail vein at 5, 15, and 30 minutes, and 1, 2, 4, 6, 8, and 24 hours after dosing. Blood samples are collected in Greiner MiniCollect K₂ EDTA tubes, placed on ice, and within 30 minutes, centrifuged at 15,000 g for 5 minutes to obtain plasma samples. All plasma samples are stored at −70° C. until analysis. Plasma samples were prepared for analysis by LC-MS/MS, and key PK parameters, including terminal half-life (t 112), initial plasma concentration (C₀), area under the plasma concentration vs. time curve (AUC), volume of distribution at steady-state (Vₛₛ), total plasma clearance (CLₚ), and mean residence time (MRT), were measured.

Microsomal stability of M1i-124 was determined using both human (mixed gender) and mouse (male CD-1) liver microsomes. Samples at a final concentration of 2μg/mL M1i-124 were prepared in 25 mM potassium phosphate buffer with liver microsomes of each species at a final concentration of 0.5 mg/mL. The enzyme reaction was initiated by the addition of NADPH reagent at a final concentration of 1 mM. For negative control samples, NADPH reagent was not added. All samples were incubated at 37° C. on a 50-RPM orbital shaker, and an aliquot was removed at pre-determined time points (0, 5, 10, 15, 30, and 60 30 minutes). Samples were precipitated with three volume of acetonitrile containing propranolol as internal standard, and centrifuged for 10 mM at 2000 g before LC/MS/MS analysis of the supernatant solutions. Metabolic products from exposure to liver microsomes were identified by LC/MS/MS.

Cytochrome P450 inhibition assays were conducted in 0.2 mg/mL mix-gender, pooled human liver microsomes (HLM), 100 mM potassium phosphate buffer, and a 2 mM NADPH cofactor. Individual CYP450 isoforms (CYP1A2, CYP2C9, CYP2C19, CYP2D6, CYP3A4, CYP2A6, CYP2B6, CYP2C8, and CYP2E1) were investigated where isoform-specific probe substrates (at concentrations near the specific enzyme's Km) are incubated individually with HLM and the test compound or a corresponding positive control inhibitor. The organic solvent was DMSO, maintained at a final concentration no greater than 1% in the assay. After incubation at 37° C. for 15-30 min, the reaction was then stopped by the addition of two volumes of acetonitrile containing internal standard. The resulting samples were centrifuged and the supernatants collected and analyzed for metabolites of the probe substrates by LC-MS/MS at each of the test compound concentrations. A decrease in the formation of the metabolites compared to vehicle control (negative control) was used to calculate percent inhibition at each concentration or an IC50 value (test compound concentration which produces 50% inhibition).

M1i-124 was screened for possible off-target effects using the Eurofins SAFETYscan 78-target panel, which screens for target activation/inhibition of 78 different high-impact molecules including GPCRs, ion channels, kinases, metabolic and other non-kinase enzymes, hormone receptors, and transporters. Assay details are available through Eurofins.

### Statistics

All values are represented as mean±SEM or ±SD as indicated in figure legends. Data were analyzed and statistics performed using GraphPad Prism software. Student's t-test was used for comparing two groups and

ANOVA test was used for comparing three or more groups. Dose-response curves were fitted using the following equation: Y=bottom+(top-bottom)/(1+10\hat{ }(X-LogIC50), with the top and bottom representing the plateaus in the units of Y axis. In vivo tumor growth curve was evaluated using two-way ANOVA and Sidak's multiple comparison tests.

### METHODS REFERENCES

1. Lucas P C, Yonezumi M, Inohara N, McAllister-Lucas L M, Abazeed M E, Chen F F, et al. Bcl10 and MALT1, independent targets of chromosomal translocation in malt lymphoma, cooperate in a novel NF-kappa B signaling pathway. J Biol Chem. 2001;276(22):19012-9.

2. Inohara N, Koseki T, Lin J, del Peso L, Lucas P C, Chen F F, et al. An induced proximity model for NF-kappa B activation in the Nodl/RICK and RIP signaling pathways. J Biol Chem. 2000;275(36):27823-31.

3. O'Shannessy D J, Brigham-Burke M, Soneson K K, Hensley P, and Brooks I. Determination of rate and equilibrium binding constants for macromolecular interactions using surface plasmon resonance: use of nonlinear least squares analysis methods. Anal Biochem. 1993;212(2):457-68.

It has also been found that M11-124 potently induces the degradation of both Bcl10 and MALT1 in TMD8 cells, in a time- and dose-dependent manner. We confirmed similar loss of Bcl10 and MALT1 proteins in another ABC-DLBCL cell line, OCI-Ly3. After several days of M1i-124 treatment, these cells completely lose all detectable MALT1 protein.

In view of the many possible embodiments to which the principles of the disclosed invention may be applied, it should be recognized that the illustrated embodiments are only preferred examples of the invention and should not be taken as limiting the scope of the invention.

1. A method comprising administering to a subject in need of, or has been recognized as being in need of, treatment with a MALT1 inhibitor, a therapeutically effective amount of a compound, or a pharmaceutically acceptable salt thereof, having a structure of: 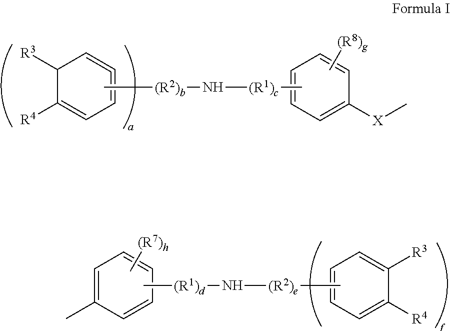
wherein X is (—CH₂—)_{y}, (—CH(R¹³)—)_{y}, (—C(R¹⁴)(R¹⁵)—)_{y}, —O—, —S(02)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R¹⁰)—)ₓ, (—C(R)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d ore is 1; and
provided that if —(R²)_{b}— is H, then a is 0, and if —(R²)ₑ— is H, then f is 0.

2. The method of claim 1, wherein the subject has a disease or condition in which MALT1 function is deregulated.

3. A method for treating a T- and B-cell malignancy, a carcinoma, an autoimmune disease, an inflammatory disease, or an allergy in a subject, comprising administering to the subject a therapeutically effective amount of a compound, or a pharmaceutically acceptable salt thereof, having a structure of: 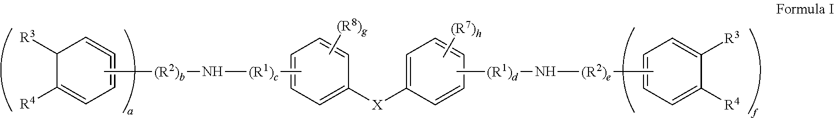
wherein X is (—CH₂—)_{y}, (—CH(R¹³)—)_{y}, (—C(R¹⁴)(R¹⁵)—)_{y}, —O—, —S(02)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R¹⁰)—)ₓ, (—C(R)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d ore is 1; and
provided that if —(R²)_{b}— is H, then a is 0, and if —(R²)ₑ— is H, then f is 0, thereby treating the T- and B-cell malignancy, the carcinoma, the autoimmune disease, the inflammatory disease, or the allergy.

4. The method of claim 3, wherein the T- and B-cell malignancy is lymphoma.

5. The method of claim 4, wherein the lymphoma is Activated B Cell subtype of Diffuse Large B Cell Lymphoma (ABC-DLBCL), Mantle Cell Lymphoma (MCL), Chronic Lymphocytic Leukemia (CLL), Acute T-cell Leukemia/Lymphoma (ATLL), Cutaneous T-cell Lymphoma (CTCL), Sezary Syndrome, or peripheral T-cell Lymphoma (PTCL).

6. The method of claim 3, wherein the autoimmune disease or the inflammatory disease is psoriasis, multiple sclerosis, rheumatoid arthritis, Sjogren's syndrome, colitis, systemic lupus erythematosus, graft-versus-host disease (GVHD), or asthma.

7. The method of claim 3, wherein the carcinoma is breast cancer, pancreatic cancer, melanoma, or glioblastoma.

8. The method of claim 2, wherein the disease is leukemia.

9. The method of claim 3, wherein at least one of the 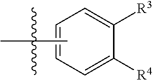
groups of formula I is

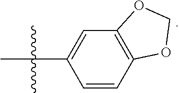 [SMILES: *c1ccc2c(c1)OCO2]

10. The method of claim 3, wherein the 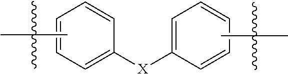
group of formula I is 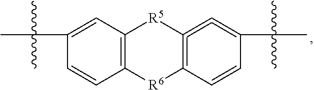
wherein R⁵ and R⁶ are each independently —CH₂ —, —O—or N.

11. The method of claim 3, wherein at least one of R¹ or R² is —S(O)₂—.

12. The method of claim 3, wherein at least one of R¹ or R² is (—CH₂—)ₓ, wherein x is 1.

13. The method of claim 3, wherein X is —CH₂—.

14. The method of claim 3, wherein —(R¹)ₑ— is —S(O)₂—, and —(R¹)_{d}— is —S(O)₂—.

15. The method of 9 claim 3, wherein —(R²)_{b}— is —CH₂—, —(R¹)ₑ— is —S(O)₂—, X is —CH₂—, —(R¹)_{d}— is —S(O)₂—, and —(R²)ₑ— is —CH₂—.

16. The method of claim 3, wherein at least one of R³ and R⁴ is alkoxy.

17. The method of claim 3, wherein each of a, b, c, d, e, and f is 1.

18. The method of claim 3, wherein each of b, c, d, and e is 1.

19. The method of claim 3, wherein at least one of a or f is 0.

20. The method of claim 3, wherein —(R²)_{b}— is H, —(R²)_{d}— is H, —(R¹)ₑ— is —S(O)₂—, and —(R²)_{b}— is —S(O)₂.

21. The method of claim 3, wherein R³ and R⁴ together form a fused bicyclic structure with the benzene ring that is adjacent to R³ and R⁴, and the fused bicyclic structure includes at least one heteroatom.

22. A pharmaceutical composition comprising a pharmaceutically acceptable additive and a compound, or a pharmaceutically acceptable salt thereof, having a structure of: 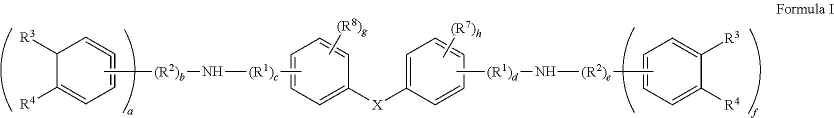
wherein X is (—CH₂—)_{y}, (—CH(R¹³)—)_{y}, (—C(R¹⁴)(R¹⁵)—)_{y}, —O—, —S(02)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R¹⁰)—)ₓ, (—C(R)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d ore is 1; and
provided that if —(R²)_{b}— is H, then a is 0, and if —(R²)ₑ— is H, then f is 0, wherein the pharmaceutical composition is in a unit dosage form.

23. A compound, or a pharmaceutically acceptable salt thereof, having a structure of: 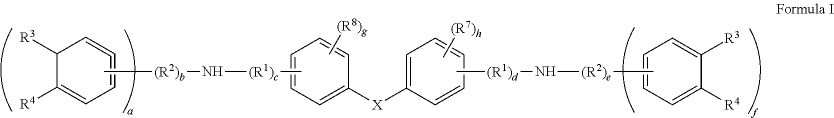
wherein X is (—CH₂—)_{y}, (—CH(R¹³)—)_{y}, (—C(R¹⁴)(R¹⁵)—)_{y}, —O—, —S(02)—, cycloalkyl, an alkynyl, or a single bond, wherein y is 1 or 2 and each R¹³, R¹⁴ and R¹⁵ is independently an alkyl, substituted alkyl, halogen, cycloalkyl, or oxo; or X together with the two phenyl groups forms a fused polycyclic structure with the two benzine rings adjacent to X;
each R¹ and R² is independently H, (—CH₂—)ₓ, (—CH(R¹⁰)—)ₓ, (—C(R)(R¹²)—)ₓ, —S(O)₂—, —C(O)—, or —NHC(O)—, wherein x is 1 or 2 and each R¹⁰, R¹¹ and R¹² is independently an alkyl;
each R³ and R⁴ is independently H, alkyl, substituted alkyl, alkoxy, or substituted alkoxy, or R³ and R⁴ together form a fused bicyclic structure with the benzene ring that is adjacent to R³ and R⁴;
each R⁷ and R⁸ is independently an alkyl; and
each of a, b, c, d, e, f, g, and h are independently 0 or 1, provided at least one of b or c is 1 and at least one of d ore is 1; and
provided that if —(R²)_{b}— is H, then a is 0, and if —(R²)ₑ— is H, then f is 0, and provided that the compound is not

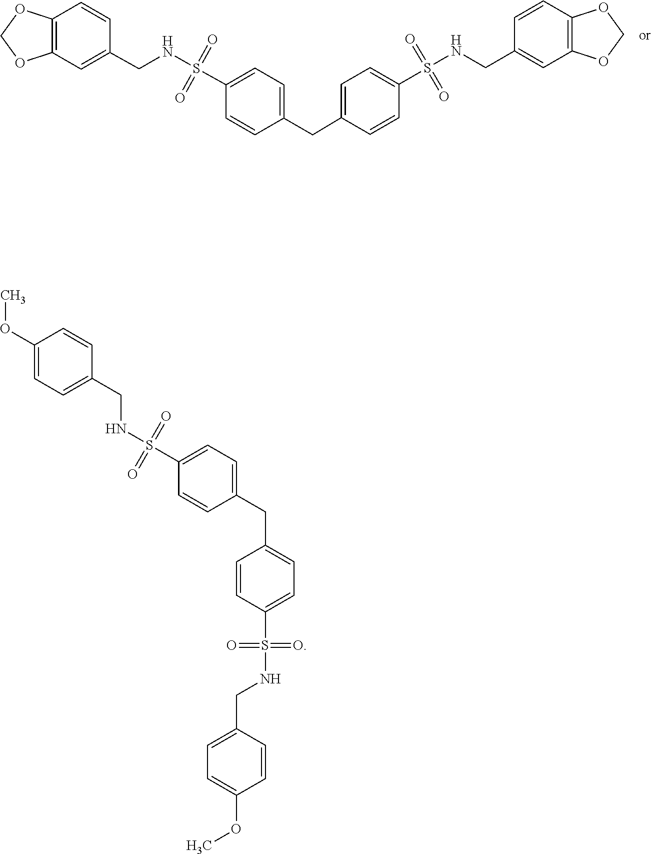 [SMILES: O=S(=O)(NCc1ccc2c(c1)OCO2)c1ccc(Cc2ccc(S(=O)(=O)NCc3ccc4c(c3)OCO4)cc2)cc1 ; COc1ccc(CNS(=O)(=O)c2ccc(Cc3ccc(S(=O)(=O)NCc4ccc(OC)cc4)cc3)cc2)cc1]

24. A method comprising administering to a subject in need of, or has been recognized as being in need of, treatment with a MALT1 inhibitor, a therapeutically effective amount of a compound, or a pharmaceutically acceptable salt thereof, having a structure of: 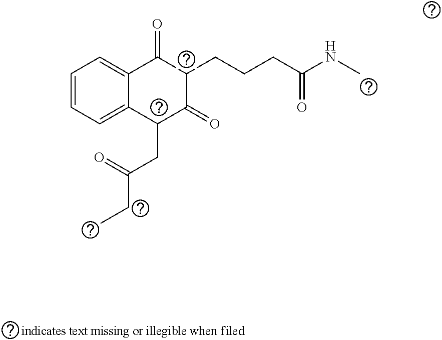
wherein R₁ and R₂ are each independently aryl, substituted aryl, aralkyl, or substituted aralkyl.
